(19) United States
(12) Patent Application Publication
(10) Pub. No.: US 2024/0294572 A1
(43) Pub. Date: Sep. 5, 2024

(54) Antimicrobial Peptides

(71) Applicant: Board of Regents of the University of Nebraska, Lincoln, NE (US)

(72) Inventors: Guangshun Wang, Omaha, NE (US); Radha M. Golla, Elkhorn, NE (US); Jayaram Lakshmaiah Narayana, Omaha, NE (US)

(21) Appl. No.: 18/278,865

(22) PCT Filed: Apr. 22, 2022
(86) PCT No.: PCT/US22/25930

§ 371 Date: Aug. 25, 2023

Related U.S. Application Data
(60) Provisional application No. 63/178,637, filed on Apr. 23, 2021.

(51) Int. Cl. C07K 7/08; A61K 38/00; A61K 47/54; A61P 31/04; C07K 5/11

(52) U.S. Cl. CPC C07K 7/08; A61K 47/542; A61P 31/04; C07K 5/1019; A61K 38/00

(57) ABSTRACT

Antimicrobial peptides and methods of use are provided.

This application claims priority under 35 U.S.C. § 119(e) to U.S. Provisional Patent Application No. 63/178,637, filed on Apr. 23, 2021. The foregoing application is incorporated by reference herein.

This invention was made with government support under R01 AI105147 and R01 GM138552 awarded by the National Institutes of Health. The government has certain rights in the invention.

### FIELD OF THE INVENTION

The present invention relates to the field of antimicrobial peptides and the treatment of microbial infections. More specifically the invention provides anti-microbial peptides and methods of using such peptides for the inhibition, treatment, and/or prevention of microbial infections.

### BACKGROUND OF THE INVENTION

Like plants and insects, humans also deploy host defense antimicrobial peptides (AMPs). These innate immune peptides play a critical role in warding off invading pathogenic bacteria, viruses, fungi, and parasites (Mookherjee, et al. (2020) Nat. Rev. Drug Discovery 19:311-332). Some of these peptides are able to eliminate the ESKAPE pathogens (Enterococcus faecium, Staphylococcus aureus, Klebsiella pneumoniae, Acinetobacter baumannii, Pseudomonas aeruginosa, and Enterobacter species) in both planktonic and biofilm forms (Rice, L. B. (2008) J. Infect. Dis., 197:1079-81). In addition, AMPs also regulate the immune systems by neutralization of endotoxin (lipopolysaccharides), association with cell receptors, and recruitment of immune cells to clear infection (Nizet, et al. (2001) Nature 414:454-7; Pütsep, et al. (2002) Lancet 360:1144-1149; Scott, et al. (2002) J. Immunol., 169:3883-91). As of Jan. 8, 2021, 141 human AMPs have been registered in the antimicrobial peptide database (aps.unmc.edu/AP; Wang, G. (2014) Pharmaceuticals 7:545-594). The major families of human AMPs are defensins, cathelicidins, histatins, and lactoferricin. Of note, there are numerous human defensins but only one cathelicidin gene. Also, some known polypeptides possess antimicrobial activity, including cytokines, neuropeptides, and β-amyloid peptides (Scott, et al. (2002) J. Immunol., 169:3883-91; Wang, G. (2014) Pharmaceuticals 7:545-594).

Unlike horse, sheep, and pigs, humans have only one cathelicidin gene (Agerberth, et al. (1995) Proc. Natl. Acad. Sci., 92:195-9). Interestingly, human cathelicidin can be processed into different molecular forms. LL-37, one of the mature AMPs, is most widely studied (Dürr, et al. (2006) Biochim. Biophys. Acta, Biomembr., 1758:1408-1425; Vandamme, et al. (2012) Cell. Immunol., 280:22-35; Wang, et al. (2014) Biochim. Biophys. Acta, Biomembr., 1838:2160-2172). Another form, ALL-38, which contains one extra alanine at the N-terminus (amino terminus), is released in the human reproduction system, probably to protect the fertilized egg from microbial infection (Sorensen, et al. (2003) J. Biol. Chem., 278:28540-6). ALL-38 is one residue shorter than FALL-39 originally predicted prior to the isolation of LL-37 (Agerberth, et al. (1995) Proc. Natl. Acad. Sci., 92:195-9). Recently, TLN-58, an even longer form, is found from a diseased state (Murakami, et al. (2017) J. Invest. Dermatol. 137:322-331). Other processed forms of human cathelicidin are also possible. For instance, an alternative form detected in fat cells has not been sequenced (Zhang, et al. (2015) Science 347:67-71). In addition, human LL-37 can be cleaved into a variety of fragments on human skin, further enriching the peptide reservoir of the human cathelicidin (Murakami, et al. (2004) J. Immunol., 172:3070-7).

The interest in identifying the antimicrobial regions of human LL-37 resulted in numerous artificial peptides. Many active fragments involve the central region of LL-37 (Braff, et al. (2005) J. Immunol., 174:4271; Nagant, et al. (2012) Antimicrob. Agents Chemother., 56:5698-708; den Hertog, et al. (2006) Biol. Chem., 387:1495-502; Nan, et al. (2012) Peptides 35:239-47; Nell, et al. (2006) Peptides 27:649-60; Oren, et al. (1999) Biochem. J., 341:501-13; Sieprawska-Lupa, et al. (2004) Antimicrob. Agents Chemother., 48:4673-9; Sigurdardottir, et al. (2006) Antimicrob. Agents Chemother., 50:2983-9; Turner, et al. (1998) Antimicrob. Agents Chemother., 42:2206-14; Li, et al. (2006) J. Am. Chem. Soc., 128:5776-85). The major antimicrobial region (residues 17-32) of LL-37 was identified by two-dimensional (2D) NMR spectroscopy using the TOCSY-trim technology (Li, et al. (2006) J. Am. Chem. Soc., 128:5776-85). The TOCSY-trim technology enables the removal of those amino acids that do not or weakly associate with bacterial membrane-mimicking micelles. An N-terminally glycine-appended version of this major antibacterial peptide (FK-16) is referred to as GF-17 (Wang, et al. (2012) Antimicrob. Agents Chemother., 56:845-856). GF-17 is demonstrated to have different activities, including antibacterial, anticancer, antibiofilm, anti-HIV, anti-Zika, anti-influenza, anti-Ebola virus, and spermicidal activities (Li, et al. (2006) J. Am. Chem. Soc., 128:5776-85; Wang, et al. (2012) Antimicrob. Agents Chemother., 56:845-856; Mishra, et al. (2016) ACS Med. Chem. Lett., 7:117-121; Wang, et al. (2008) Antimicrob. Agents Chemother., 52:3438-3440; He, et al. (2018) Front. Immunol., 9:722; Tripathi, et al. (2015) PLOS One 10:e0133454; Yu, et al. (2020) iScience 23:100999; Kiattiburut, et al. (2018) Hum. Reprod., 33:2175-2183). GF-17 eliminates both Gram-positive and negative pathogens (Wang, et al. (2012) Antimicrob. Agents Chemother., 56:845-856; Wang, et al. (2018) Adv. Biosyst., 2:1700259). It is possible to convert GF-17 to narrow-spectrum antimicrobial molecules. The partial incorporation of D-amino acids into GF-17 led to GF-17d3, which is active against Escherichia coli (E. coli) and A. baumannii but not other bacteria that were tested (Wang, et al. (2018) Adv. Biosyst., 2:1700259). It was also possible to make the peptide only inhibitory to Gram-positive pathogens such as methicillin-resistant S. aureus (MRSA) (Wang, et al. (2018) Adv. Biosyst., 2:1700259). Alternatively, via sequence truncation of GF-17, KR12 (residues 18-29 of LL-37) was obtained, which is the minimal antibacterial sequence of LL-37 that inhibits E. coli but not MRSA (Wang, G. (2008) J. Biol. Chem., 283:32637-32643; Mishra, et al. (2013) RSC Adv., 3:19560). These results suggest different sequence requirements for cationic peptides to inhibit Gram-positive and Gram-negative pathogens.

To develop the potential medical use of LL-37, GF-17 was converted into a stable, selective, and potent peptide 17BIPHE2 against the ESKAPE pathogens in planktonic or biofilm forms (Wang, et al. (2018) Adv. Biosyst., 2:1700259; Wang, et al. (2014) ACS Chem. Biol., 9:1997-2002; Narayana, et al. (2019) Biochim. Biophys. Acta, Biomembr., 1861:1592-1602). Other peptides based on FK-13 or KR12, the core and minimal antimicrobial peptides of LL-37, were synthesized. These range from simple amino acid substitutions to a more sophisticated cyclization of KR12 (Rajasekaran, et al. (2017) Biochim. Biophys. Acta, Biomembr., 1859:722-733; Jacob, et al. (2013) J. Pept. Sci., 19:700-7; Gunasekera, et al. (2020) Front. Microbiol., 11:168; Wang, et al. (2019) Adv. Exp. Med. Biol., 1117: 215-240). Despite these advances, antimicrobial peptides activities are often compromised in the presence of physiological salts, pH, and serum. Furthermore, longer peptide sequences are cost prohibitive to manufacture and invite the development of antimicrobial resistance. Accordingly, there is a strong need for improved antimicrobial peptides.

### SUMMARY OF THE INVENTION

In accordance with one aspect of the instant invention, antimicrobial peptides are provided. In certain embodiments, the peptides are lipidated peptides (e.g., antimicrobial lipopeptides). In certain embodiments, the peptides are conjugated to a fatty acid, particularly a saturated fatty acid. Compositions comprising at least one antimicrobial peptide of the instant invention and at least one pharmaceutically acceptable carrier are also provided. The compositions may further comprise at least one other antimicrobial compound (e.g., antibiotic). Medical devices and medical implants comprising an antimicrobial peptide (e.g., on its surface) are also provided, along with methods of making the same.

In accordance with another aspect of the instant invention, methods for inhibiting, treating, and/or preventing a microbial infection in a subject are provided. The methods comprise administering (e.g., topically) to a subject at least one antimicrobial peptide of the instant invention, particularly as a composition with a carrier or attached (e.g., covalently or noncovalently) to the surface of a medical device or implant. In a particular embodiment, the methods further comprise the administration at least one other antimicrobial treatment, such as the administration of at least one additional antibiotic.

### BRIEF DESCRIPTIONS OF THE DRAWING

FIG. 1 provides a mass spectrometry analysis of mouse serum proteins binding to the L-and D-forms of C10-KR8 immobilized to beads. Heat map representation of the abundances of 20 significantly up-regulated and down-regulated proteins (PEAKS Q) bound to C10-KR8, C10-KR8d, and the peptide uncoupled control samples (left to right) after unsupervised hierarchical clustering.

FIG. 2 shows the protease stability of the LL-37 derived C10-KR8 detected by SDS-PAGE. Top: C10-KR8 (L-form): untreated peptide (arrow); peptide treated with 2: bovine chymotrypsin; 3: porcine pancreas trypsin; 4: S. aureus V8 protease; 5: fungal proteinase K; or 6: porcine pancreas elastase for 1 day. Peptide treated with 7: chymotrypsin; 8: trypsin; 9: S. aureus V8 protease; or 10: porcine pancreas elastase for 3 hours. Bottom: C10-KR8d (D-form): untreated peptide (arrow); peptide treated with 2: chymotrypsin; 3: trypsin; 4: S. aureus V8 protease; 5: proteinase K; or 6: porcine pancreas elastase for 1 day. Peptide treated with 7: chymotrypsin; 8: trypsin; 9: S. aureus V8 protease; or 10: porcine pancreas elastase for 3 hours.

FIGS. 3A and 3B show the membrane permeation and depolarization by the miniature LL-37 peptide C10-KR8d. FIG. 3A: Effects of C10-KR8d, 17BIPHE2, and daptomycin on membrane integrity based on the fluorescence of a membrane nonpermeable dye propidium iodide. S. aureus USA300 LAC treated with 12.5 μM of peptides and relative fluorescence intensity was recorded with time. 17BIPHE2 and daptomycin are used as positive controls, while rifamycin was included as a negative control for the assay. FIG. 3B: Effects of peptides on membrane depolarization of the mid log phase S. aureus USA300 resuspended in PBS and glucose energized for 15 minutes at 37° C. C10-KR8d, 17BIPHE2, and daptomycin (12.5 μM) were added to respective wells at post-dye equilibration pause cycle and the fluorescence was monitored for 40 minutes. The detergent Triton™ -100 (0.1%) was included as a positive control. FIG. 3C shows the effects of peptides on membrane depolarization of the mid-log phase S. aureus USA300 re-suspended in PBS and glucose energized for 15 minutes at 37° C. C10-KR8d, 17BIPHE2 and daptomycin at 3.1, 6.25, 12.5, and 25 μM were added to respective wells at post-dye equilibration pause cycle and the fluorescence was monitored for 40 minutes. The detergent Triton™ X-100 (0.1%) was included as a positive control. The same trend at different treated concentrations was observed. The panel at 12.5 μM is the same as FIG. 3B.

FIGS. 4A and 4B provide circular dichroism (CD) spectra of a highly selective lipopeptide C10-KR8 (L-form) and its D-form C10-KR8d (made of D-amino acids) in PBS buffer (FIG. 4A) and in the presence of SDS micelles (FIG. 4B) at pH 7 and 25° C. The concentration of the peptide in all spectra was 1 mM and the peptide:SDS molar ratio was 1:60.

FIG. 5 shows the resistance development of S. aureus USA300 LAC to C10-KR8d during 14 passages in the presence of sub-MIC of the peptide. The figure represents the average of three independent experiments.

FIG. 6A shows the toxicity to HaCaT cells of the L and D-forms of the miniature lipoLL-37 peptides obtained from the 2D molecular array. FIG. 6B shows the toxicity of C10-KR8d to mice for 5 days. Toxicity was assessed using female C57BL/6 mice (n=3) by intraperitoneal administration of C10-KR8d into three testing mouse groups at an increasing amount of the peptide dose of 10, 20, and 40 mg/kg, respectively. A fourth control group was treated with vehicle.

FIG. 7 shows the systemic efficacy of C10-KR8d in neutropenic mice infected with 2×10⁶ CFU of S. aureus USA300 LAC. Two hours post infection, the peptide group was treated (i.p., 5 mg/kg, n=8) with a single dose of C10-KR8d while the control infected group was untreated. The CFU difference is significant when p value<0.05 and ns represents no significance. The statistics was determined using Mann-Whitney test in the GraphPad Prism 7.0.

FIGS. 8A-8E show the biofilm inhibition capability of C10-KR8d in a catheter-associated biofilm model challenged with S. aureus USA300 LAC. FIG. 8A: A single dose treatment with C10-KR8d (250 μg) into the implanted catheter lumen and surrounding tissues (50 μL/site; into lumen, four sides of the catheter) of mice (n=4). The catheter alone animal group without bacterial infection was included as a control. The CFU of each mouse represents individual points, and error bars represent the deviation within the experimental group. Starred CFU differences (*) with p<0.05 were considered statistically significant within groups as determined by the Mann-Whitney test (GraphPad Prism 7). ELISA quantification of the levels of MCP-1 (FIG. 8B), IL-17A (FIG. 8C), IL-10 (FIG. 8D), and TNF-α (FIG. 8E) in the surrounding tissues of catheters embedded in mice with and without treatment with C10-KR8d are also provided.

### DETAILED DESCRIPTION OF THE INVENTION

Herein, new LL-37 based peptides were synthesized that are shorter than and more robust than previous versions. A library of new lipopeptides was synthesized by conjugating numerous short KR12 peptides, including both forms made of L-and D-amino acids, with fatty acids at varying chain lengths. On the basis of antibacterial and hemolytic assays, peptides with excellent antimicrobial potency and high cell selectivity were identified. Robust antimicrobial activity in vitro against both Gram-positive and negative bacteria was observed. Moreover, the peptides also effectively tolerated the effects of media conditions such as salts, pH and serum. Peptide conformation, mechanism of action, and serum protein binding by mass spectrometry were also characterized. Proteomic studies reveal far fewer serum proteins that bind to the D-form than the L-form peptide. Additionally, the peptide targets bacterial membranes to become helical, making it difficult for bacteria to develop resistance in a multiple passage experiment. Indeed, methicillin-resistant Staphylococcus aureus(MRSA) did not develop resistance to the optimal peptide C10-KR8d in a multiple passage experiment, although multiple genes are responding to it when treated at a sublethal peptide concentration. The in vivo efficacy of the peptide was also demonstrated using both topical and systemic murine models. In addition, this designer peptide prevents bacterial biofilm formation in a catheter-associated mouse model. Meanwhile, C10-KR8d also recruits cytokines to the vicinity of catheters to clear infection. Accordingly, new peptides are provided with both robust antimicrobial, antibiofilm, and immune modulation activities which are effective against antibiotic-resistant bacteria. The lipopeptides of the instant invention demonstrate superiority over other antimicrobial peptides by demonstrating systemic efficacy, a shorter amino acid sequence, and/or superior bacterial species/target specificity.

In accordance with the instant invention, antimicrobial peptides are provided. In a particular embodiment of the instant invention, the peptide comprises a truncation of the amino acid sequence KRIXQRIKDFLR (SEQ ID NO: 8), wherein X is W or V. In a particular embodiment of the instant invention, the peptide comprises a truncation of the amino acid sequence KRIWQRIKDFLR (SEQ ID NO: 2). In certain embodiments, the peptide comprises the 4, 5, 6, 7, 8, 9, 10, or 11 N-terminal amino acids of the amino acid sequence KRIWQRIKDFLR (SEQ ID NO: 2) or KRIXQRIKDFLR (SEQ ID NO: 8), wherein X is W or V. In certain embodiments, the peptide comprises the 6, 7, 8, 9, or 10 N-terminal amino acids of the amino acid sequence KRIWQRIKDFLR (SEQ ID NO: 2) or KRIXQRIKDFLR (SEQ ID NO: 8), wherein X is W or V. In certain embodiments, the peptide comprises the 8, 9, or 10 N-terminal amino acids of the amino acid sequence KRIWQRIKDFLR (SEQ ID NO: 2) or KRIXQRIKDFLR (SEQ ID NO: 8), wherein X is W or V. In certain embodiments, the peptide comprises the amino acid sequence KRIW (SEQ ID NO: 6) or KRIV (SEQ ID NO: 9). In certain embodiments, the peptide comprises the amino acid sequence KRIWQR (SEQ ID NO: 5) or KRIVQR (SEQ ID NO: 10). In certain embodiments, the peptide comprises the amino acid sequence KRIWQRIK (SEQ ID NO: 4) or KRIVQRIK (SEQ ID NO: 11). In certain embodiments, the peptide comprises the amino acid sequence KRIWQRIKDF (SEQ ID NO: 3) or KRIVQRIKDF (SEQ ID NO: 12).

In certain embodiments, the peptides of the instant invention have fewer than 12 amino acids, fewer than 11 amino acids, fewer than 10 amino acids, fewer than 9 amino acids, fewer than 8 amino acids, fewer than 7 amino acids, fewer than 6 amino acids, or fewer than 5 amino acids. In certain embodiments, the peptides of the instant invention have more than 3 amino acids, more than 4 amino acids, more than 5 amino acids, more than 6 amino acids, more than 7 amino acids, more than 8 amino acids, more than 9 amino acids, or more than 10 amino acids. In certain embodiments, the peptides of the instant invention are about 4 to about 11 amino acids in length, about 6-10 amino acids in length, about 8-10 amino acids in length, or about 8 or 10 amino acids in length.

The amino acid sequence of the antimicrobial peptide of the instant invention may have at least 75%, at least 80%, at least 85%, at least 90%, at least 95%, at least 97%, at least 99%, or 100% homology or identity with any peptide described herein (e.g., SEQ ID NOs: 2-12) or any one of the above sequences, particularly at least 90% homology or identity (e.g., the sequence may contain additions, deletions, and/or substitutions). In certain embodiments, the W sat position 4 of the peptides of the instant invention is replaced with a V. In a particular embodiment, the antimicrobial peptide of the instant invention may extend beyond any peptide described herein (e.g., SEQ ID NOs: 2-12) or any one of the above sequences at the amino and/or carboxyl terminus by 1, 2, 3, 4, 5, 6, 7, 8, 9, or 10 amino acids, particularly by 1, 2, 3, 4, or 5 amino acids, by 1, 2, or 3 amino acids, by 1 or 2 amino acids, or by 1 amino acid. In yet another embodiment, the antimicrobial inhibitory peptides of the instant invention may also be in reverse orientation (i.e., the sequence from amino terminus to carboxyl terminus is reversed).

In a particular embodiment, the antimicrobial peptide of the instant invention comprises any peptide described herein (e.g., SEQ ID NOs: 2-12) or any one of the above sequences and a terminal cysteine. In a particular embodiment, the cysteine is at the amino terminus. In a particular embodiment, the cysteine is at the carboxyl terminus. The presence of the cysteine provides functionality to allow the covalent linkage of the antimicrobial peptide to, for example, a solid surface (e.g., a medical implant).

As stated hereinabove, the antimicrobial peptide of the instant invention may contain substitutions for the amino acids of the provided sequence. These substitutions may be similar to the amino acid (i.e., a conservative change) present in the provided sequence (e.g., an acidic amino acid in place of another acidic amino acid, a basic amino acid in place of a basic amino acid, a hydrophobic amino acid in place of a hydrophobic amino acid, a polar amino acid for a polar amino acid, etc.). The substitutions may also comprise amino acid analogs and mimetics. In a particular embodiment, the substitutions are predicted to promote and/or retain helicity or helix formation. In certain embodiments, the Trp (W) at position 4 of the peptides is replaced with a Val (V).

The antimicrobial peptide of the instant invention may have capping, protecting and/or stabilizing moieties at the C-terminus and/or N-terminus. Such moieties are well known in the art and include, without limitation, amidation and acetylation. In a particular embodiment, the peptides of the instant invention are amidated (e.g., at the C-terminus). The peptide template may also be glycosylated at any amino acid (i.e., a glycopeptide). In particular, these peptides may be PEGylated to improve druggability. The number of the PEG units (NH₂(CH₂CH₂O)CH₂CH₂CO) may vary, for example, from 1 to about 50.

The antimicrobial peptide of the instant invention may also comprise at least one D-amino acid instead of the native L-amino acid. The antimicrobial peptide may comprise only D-amino acids. In a particular embodiment, the antimicrobial peptides comprise D-amino acids which are spaced apart by about 1, 2, 3, and/or 4 (e.g., 3) consecutive L-amino acids.

The antimicrobial peptides of the instant invention may contain at least one derivative of standard amino acids, such as, without limitation, fluorinated residues or nonstandard amino acids (e.g., beta-amino acids). In yet another embodiment, the peptide may also be circulated head to tail or locally involving a few residues.

In certain embodiments, the peptides of the instant invention are lipidated. In certain embodiments, the peptides are conjugated to a lipid (e.g., fatty acids, triglyceride, diglyceride, monoglyceride, phospholipid, and the like), either directly or via a linker. In certain embodiments, the peptides are conjugated to a fatty acid, either directly or via a linker. In certain embodiments, the lipid (e.g., fatty acid) is conjugated to the N-terminus and/or C-terminus of the peptide. In certain embodiments, the lipid (e.g., fatty acid) is conjugated to the N-terminus of the peptide. In certain embodiments, the lipid (e.g., fatty acid) is conjugated to the peptide via an amid bond (e.g., via the reaction of the amine group at the N-terminus of the peptide and the carboxylic acid of the fatty acid). The fatty acid can be saturated or unsaturated. In certain embodiments, the fatty acid is saturated. In certain embodiments, the fatty acid is a saturated or unsaturated C4-C20 fatty acid, a C4-C18 fatty acid, a C4-C16 fatty acid, a C6-C14 fatty acid, a C7-C14 fatty acid, a

C7-C13 fatty acid, a C8-C12 fatty acid, a C7-C12 fatty acid, a C7-C11 fatty acid, a C8-C11 fatty acid, a C8-C10 fatty acid, a C8 fatty acid, a C9 fatty acid, or a C10 fatty acid. In certain embodiments, the fatty acid is at least 4, 5, 6, 7, 8, 9, 10, 11, 12, 13, or 14 carbons in length. In certain embodiments, the fatty acid is fewer than 14, 13, 12, 11, 10, 9, 8, 7, 6, 5, or 4 carbons in length.

In certain embodiments, the peptide of the instant invention comprises the amino acid sequence KRIWQRIK (SEQ ID NO: 4) conjugated to a lipid (e.g., fatty acid). In certain embodiments, the peptide of the instant invention comprises the amino acid sequence KRIWQRIK (SEQ ID NO: 4) conjugated to a fatty acid at the N-terminus of the amino acid sequence. In certain embodiments, the amino acid sequence comprises at least one D-amino acid. In certain embodiments, the amino acid sequence comprises only D-amino acids. In certain embodiments, the Trp (W) at position 4 of the peptides is replaced with a Val (V). In certain embodiments, the fatty acid is saturated. In certain embodiments, the fatty acid is a saturated or unsaturated C6-C14 fatty acid, a C7-C13 fatty acid, a C7-C12 fatty acid, a C8-C12 fatty acid, a C8-C11 fatty acid, a C9-C11 fatty acid, a C7-C11 fatty acid, a C7-C10 fatty acid, a C8-C10 fatty acid, a C8 fatty acid, a C9 fatty acid, or a C10 fatty acid. In certain embodiments, the fatty acid is a C10 fatty acid, particularly a saturated C10 fatty acid (decanoic acid).

In certain embodiments, the peptide of the instant invention comprises the amino acid sequence KRIWQRIKDF (SEQ ID NO: 3) conjugated to a lipid (e.g., fatty acid). In certain embodiments, the peptide of the instant invention comprises the amino acid sequence KRIWQRIKDF (SEQ ID NO: 3) conjugated to a fatty acid at the N-terminus of the amino acid sequence. In certain embodiments, the amino acid sequence comprises at least one D-amino acid. In certain embodiments, the amino acid sequence comprises only D-amino acids. In certain embodiments, the Trp (W) at position 4 of the peptides is replaced with a Val (V). In certain embodiments, the fatty acid is saturated. In certain embodiments, the fatty acid is a saturated or unsaturated C6-C14 fatty acid, a C6-C13 fatty acid, a C6-C12 fatty acid, a C6-C11 fatty acid, a C6-C10 fatty acid, a C7-C12 fatty acid, a C7-C11 fatty acid, a C7-C10 fatty acid, a C7-C9 fatty acid, a C8 fatty acid, a C9 fatty acid, or a C10 fatty acid. In certain embodiments, the fatty acid is a C8 fatty acid, particularly a saturated C8 fatty acid (octanoic acid).

The present invention also encompasses compositions comprising at least one peptide of the instant invention and at least one carrier (e.g., a pharmaceutically acceptable carrier). In a particular embodiment, the composition comprises at least two peptides of the instant invention. The present invention also encompasses methods for preventing, inhibiting, and/or treating microbial infections, including microbial/bacterial biofilms. The compositions of the instant invention can be administered to an animal, in particular a mammal (e.g., a human) in order to treat, inhibit, and/or prevent a microbial (e.g., bacterial such as by E. coli, S. aureus, any the ESKAPE pathogen (including Enterococcus faecium, Staphylococcus aureus, Klebsiella pneumoniae, Acinetobacter baumannii, Pseudomonas aeruginosa, and Enterobacter species), MRSA, etc.) infection (e.g., the composition may be administered before, during, and/or after a microbial infection). The compositions of the instant invention may also comprise at least one other antimicrobial agent (e.g., an antibiotic). The additional antimicrobial agent may also be administered in a separate composition from the antimicrobial peptides of the instant invention. The compositions may be administered at the same time and/or at different times (e.g., sequentially). The composition(s) comprising at least one peptide of the instant invention and the composition(s) comprising at least one additional antibiotic may be contained within a kit.

The peptides of the present invention may be prepared in a variety of ways, according to known methods. In a particular embodiment, the antimicrobial peptides of the instant invention are chemically synthesized. For example, the peptides may be synthesized using a liquid-phase method or solid-phase method. The chemically synthesized peptides may then be purified (e.g., by HPLC).

The peptides may also be purified from appropriate sources (e.g., bacterial or animal cultured cells or tissues, optionally transformed) by immunoaffinity purification. The availability of nucleic acid molecules encoding the antimicrobial peptides enables production of the protein using in vitro expression methods and cell-free expression systems known in the art.

Larger quantities of peptides may be produced by expression in a suitable prokaryotic or eukaryotic system. For example, part or all of a DNA molecule encoding for an antimicrobial peptide may be inserted into a plasmid vector adapted for expression in a bacterial cell, such as E. coli. Such vectors comprise the regulatory elements necessary for expression of the DNA in the host cell positioned in such a manner as to permit expression of the DNA in the host cell. Such regulatory elements required for expression include promoter sequences, transcription initiation sequences and, optionally, enhancer sequences.

Peptides produced by gene expression in a recombinant prokaryotic or eukaryotic system may be purified according to methods known in the art. A commercially available expression/secretion system can be used, whereby the recombinant protein is expressed and thereafter secreted from the host cell, and readily purified from the surrounding medium. If expression/secretion vectors are not used, an alternative approach involves purifying the recombinant protein by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope or the hemaglutinin epitope. Such methods are commonly used by skilled practitioners.

Peptides of the invention, prepared by the aforementioned methods, may be analyzed and verified according to standard procedures. For example, such peptides may be subjected to amino acid sequence analysis, according to known methods.

Infections involving implanted medical devices cost billions of dollars a year. The development of preventative antimicrobial surfaces is considered as the most promising method to combat such infection. Surface coating materials include both metals (e.g., silver, zinc, copper, and zirconium) and non-metals (e.g., selenium and antibiotics). However, the effective use of metals such as silver is complicated by leaching and cytotoxicity issues, whereas a prolonged use of antibiotics results in reduced efficacy due to the emergence of multi-drug resistance pathogens. Surface coating with antimicrobial peptides is the best option to prevent biofilm formation. Antimicrobial peptides have the advantage of being potent against drug-resistant superbugs and will not leak into the surrounding tissues due to covalent immobilization. The antimicrobial peptides can be covalently immobilized to different surfaces ranging from plastics (e.g., polyethylene terephthalate) to metals (e.g., titanium).

Medical devices or implants comprising at least one peptide of the instant invention are provided, along with methods of making the same. As used herein, the term “medical device” or “medical implant” includes devices, implants, and materials that are permanently implanted and those that are temporarily or transiently present in the patient. In a particular embodiment, at least part of the exposed surface of the medical device or implant is coated with at least one antimicrobial peptide of the instant invention. In a particular embodiment, the medical device or implant comprises a plastic (e.g., polyethylene terephthalate) or a metal (e.g., titanium). In a particular embodiment, the peptide is covalently attached to the surface of the medical implant or device. The peptide may be linked directly (e.g., via a bond) to the surface of the medical device or implant or covalently attached via a linker (e.g., a crosslinker).

The peptide attached to the surface of the medical device or medical implant may be any peptide having antimicrobial activity. In a particular embodiment, the antimicrobial peptide comprises one any peptide described herein (e.g., SEQ ID NOs: 2-12) or any one of the sequences set forth herein.

The instant invention also encompasses methods of synthesizing the coated medical device or medical implant of the instant invention. In a particular embodiment, the method comprises linking a peptide of the instant invention comprising a terminal cysteine to the medical device or medical implant with a sulfhydryl reactive crosslinker (e.g., a maleimide crosslinker). The crosslinker may be reacted with the peptide first, with the medical device or medical implant first, or with both simultaneously. In a particular embodiment, the peptide is linked to a biocompatible polymer (e.g., chitosan) with a sulfhydryl reactive crosslinker (e.g., a maleimide crosslinker), wherein the biocompatible polymer is attached to the medical device or medical implant.

The term “crosslinker” refers to a molecule capable of forming a covalent linkage between compounds. In a particular embodiment, the crosslinker forms a covalent linkage (e.g., a sulfide bond) via the sulfhydryl group of the terminal cysteine of the antimicrobial peptide. Crosslinkers are well known in the art. The cross-linker may be a bifunctional, trifunctional, or multifunctional crosslinking reagent. In a particular embodiment, the crosslinker is non-biodegradable or uncleavable under physiological conditions. In a particular embodiment, the crosslinker is a maleimide crosslinker.

In accordance with another aspect of the instant invention, nucleic acid molecules encoding the peptides are provided. Nucleic acid molecules encoding the peptides of the invention may be prepared by any method known in the art such as, without limitation: (1) synthesis from appropriate nucleotide triphosphates or (2) isolation and/or amplification from biological sources. The availability of nucleotide sequence information enables preparation of an isolated nucleic acid molecule of the invention by oligonucleotide synthesis. Indeed, knowledge of the amino sequence is sufficient to determine an encoding nucleic acid molecule. Synthetic oligonucleotides may be prepared by the phosphoramidite method employed in the Applied Biosystems 38A DNA Synthesizer or similar devices. The resultant construct may be purified according to methods known in the art, such as gel electrophoresis or high performance liquid chromatography (HPLC).

Nucleic acids of the present invention may be maintained in any convenient vector, particularly an expression vector. Different promoters may be utilized to drive expression of the nucleic acid sequences based on the cell in which it is to be expressed. Antibiotic resistance markers are also included in these vectors to enable selection of transformed cells. Antimicrobial peptide encoding nucleic acid molecules of the invention include cDNA, genomic DNA, RNA, and fragments thereof which may be single-or double-stranded. Thus, this invention provides oligonucleotides having sequences capable of hybridizing with at least one sequence of a nucleic acid molecule of the present invention.

The present invention also encompasses compositions comprising at least one nucleic acid encoding a peptide of the instant invention and at least one carrier (e.g., pharmaceutically acceptable carrier).

As stated hereinabove, the present invention also encompasses compositions comprising at least one peptide of the instant invention and at least one carrier (e.g., pharmaceutically acceptable carrier). The compositions comprising the peptides of the instant invention may be conveniently formulated for administration with any pharmaceutically acceptable carrier(s). For example, the peptides may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents, or suitable mixtures thereof, particularly an aqueous solution. The concentration of the peptide in the chosen medium may be varied and the medium may be chosen based on the desired route of administration of the pharmaceutical composition. Except insofar as any conventional media or agent is incompatible with the peptide to be administered, its use in the pharmaceutical composition is contemplated.

The present invention also encompasses methods for preventing, inhibiting, and/or treating microbial infections (e.g., viral or bacterial), particularly a bacterial infection such as S. aureus infections (e.g., MRSA). The method comprises administering at least one peptide of the instant invention (optionally within a composition with a carrier) to the subject. The method may further comprise administering at least one additional antimicrobial (e.g., antibiotic). In a particular embodiment, the microbe is an antibiotic-resistant bacteria or an ESKAPE pathogen. In a particular embodiment, the microbe is in a biofilm. In a particular embodiment, the microbe is planktonic. In a particular embodiment, the microbe is selected from the group consisting of Enterococcus faecium, Staphylococcus aureus, Klebsiella pneumoniae, Acinetobacter baumannii, Pseudomonas aeruginosa, and Enterobacterspecies. The compositions of the instant invention can be administered to an animal, in particular a mammal, more particularly a human, in order to treat, inhibit, and/or prevent a microbial infection (e.g., the composition may be administered before, during, and/or after a microbial infection). The pharmaceutical compositions of the instant invention may also comprise at least one other antimicrobial agent, particularly at least one other antibiotic. The additional antimicrobial agent may also be administered in a separate composition from the antimicrobial peptides of the instant invention. The compositions may be administered at the same time and/or at different times (e.g., sequentially).

In a particular embodiment, the peptides of the instant invention are administered to the subject as a coating on a medical device or implant.

Bacterial infections that may be treated using the present methods include Gram-positive bacterial infections and Gram-negative bacterial infections. In a particular embodiment, the bacteria is a Gram-positive bacteria. In a particular embodiment, the bacteria is a staphylococcal strain. In yet another embodiment, the bacteria is Staphylococcus aureus. More particularly, the bacteria is MRSA. In a particular embodiment, the bacteria is an antibiotic-resistant bacteria or an ESKAPE pathogen. In a particular embodiment, the bacteria is selected from the group consisting of Enterococcus faecium, Staphylococcus aureus, Klebsiella pneumoniae, Acinetobacter baumannii, Pseudomonas aeruginosa, and Enterobacter species.

The peptides described herein will generally be administered to a patient as a pharmaceutical composition. The term “patient” as used herein refers to human or animal subjects. These antimicrobial peptides may be employed therapeutically, under the guidance of a physician.

The dose and dosage regimen of antimicrobial peptides according to the invention that are suitable for administration to a particular patient may be determined by a physician considering the patient's age, sex, weight, general medical condition, and the specific condition for which the antimicrobial peptides are being administered and the severity thereof. The physician may also take into account the route of administration, the pharmaceutical carrier, and the antimicrobial peptide's biological activity.

Selection of a suitable pharmaceutical composition will also depend upon the mode of administration chosen. For example, the antimicrobial peptides of the invention may be administered by direct injection or intravenously. In this instance, a pharmaceutical composition comprises the peptide dispersed in a medium that is compatible with the site of injection.

Peptides of the instant invention may be administered by any method. For example, the antimicrobial peptides of the instant invention can be administered, without limitation by injection, parenterally, subcutaneously, orally, nasally, topically, pulmonarily, rectally, vaginally, intravenously, intraperitoneally, intrathecally, intracerbrally, epidurally, intramuscularly, intradermally, intracarotidly, or other modes of administration such as controlled release devices. In a particular embodiment, the peptides (or compositions) are administered intramuscularly, subcutaneously, or to the bloodstream (e.g., intravenously). In a particular embodiment, the peptides (or compositions) are administered by injection. In a particular embodiment, the peptides (or compositions) are administered topically. The peptides (or compositions) may be directly administered (e.g., by injection) to the site of microbial infection. In general, pharmaceutical compositions and carriers of the present invention comprise, among other things, pharmaceutically acceptable diluents, preservatives, stabilizing agents, solubilizers, emulsifiers, adjuvants and/or carriers. Such compositions can include diluents of various buffer content (e.g., saline, Tris HCl, acetate, phosphate), pH and ionic strength; and additives such as detergents and solubilizing agents (e.g., Tween™ 80, polysorbate 80), anti-oxidants (e.g., ascorbic acid, sodium metabisulfite), preservatives (e.g., Thimersol, benzyl alcohol) and bulking substances (e.g., lactose, mannitol). The compositions can be incorporated into particulate preparations of polymeric compounds such as polylactic acid, polyglycolic acid, etc., or into liposomes. Such compositions may influence the physical state, stability, rate of in vivo release, and rate of in vivo clearance of components of a pharmaceutical composition of the present invention. Exemplary pharmaceutical compositions and carriers are provided, e.g., in “Remington's Pharmaceutical Sciences” by E. W. Martin (Mack Pub. Co., Easton, Pa.) and “Remington: The Science and Practice Of Pharmacy” by Alfonso R. Gennaro (Lippincott Williams & Wilkins) which are herein incorporated by reference. The compositions of the present invention can be prepared, for example, in liquid form, or can be in pill or dried powder form (e.g., lyophilized). Pharmaceutical compositions for injection are known in the art. If injection is selected as a method for administering the peptide, steps must be taken to ensure that sufficient amounts of the molecules or cells reach their target cells to exert a biological effect. Dosage forms for oral administration include, without limitation, tablets (e.g., coated and uncoated, chewable), gelatin capsules (e.g., soft or hard), lozenges, troches, solutions, emulsions, suspensions, syrups, elixirs, powders/granules (e.g., reconstitutable or dispersible) gums, and effervescent tablets. Dosage forms for parenteral administration include, without limitation, solutions, emulsions, suspensions, dispersions and powders/granules for reconstitution. Dosage forms for topical administration include, without limitation, creams, gels, ointments, salves, patches and transdermal delivery systems.

Pharmaceutical compositions containing a peptide of the present invention as the active ingredient in intimate admixture with a pharmaceutically acceptable carrier can be prepared according to conventional pharmaceutical compounding techniques. The carrier may take a wide variety of forms depending on the form of pharmaceutical composition desired for administration, e.g., intravenous, topical, oral, direct injection, intracranial, and intravitreal.

In yet another embodiment, the compositions of the present invention can be delivered in a controlled release system, such as using an intravenous infusion, an implantable osmotic pump, a transdermal patch, liposomes, or other modes of administration. In a particular embodiment, a pump may be used.

A pharmaceutical composition of the invention may be formulated in dosage unit form for ease of administration and uniformity of dosage. Dosage unit form, as used herein, refers to a physically discrete unit of the pharmaceutical composition appropriate for the patient undergoing treatment. Each dosage should contain a quantity of active ingredient calculated to produce the desired effect (e.g., curing, relieving, treating, and/or preventing the microbial infection, the symptoms of it, or the predisposition towards it) in association with the selected pharmaceutical carrier. The dosage should not be so large as to cause adverse side effects, such as unwanted cross-reactions, anaphylactic reactions, and the like. Procedures for determining the appropriate dosage unit are well known to those skilled in the art. Generally, the dosage will vary with the age, weight, condition, sex and extent of the disease in the patient and can be determined by one of skill in the art. Appropriate concentrations for alleviation of a particular pathological condition may be determined by dosage concentration curve calculations, as known in the art. The dosage can be adjusted by the individual physician in the event of any counter indications.

In accordance with the present invention, the appropriate dosage unit for the administration of peptides may be determined by evaluating the toxicity of the molecules or cells in animal models. Various concentrations of peptides in pharmaceutical composition may be administered to mice, and the minimal and maximal dosages may be determined based on the beneficial results and side effects observed as a result of the treatment. Appropriate dosage unit may also be determined by assessing the efficacy of the antimicrobial peptide treatment in combination with other standard drugs. The dosage units of antimicrobial peptide may be determined individually or in combination with each treatment according to the effect detected.

The pharmaceutical composition comprising the peptides may be administered at appropriate intervals until the pathological symptoms are reduced or alleviated, after which the dosage may be reduced to a maintenance level. The appropriate interval in a particular case would normally depend on the condition of the patient. In a particular embodiment, the method comprises at least two administrations of the peptides or compositions.

The instant also encompasses delivering the peptides of the instant invention to a cell in vitro (e.g., in culture). The peptide may be delivered to the cell in at least one carrier.

### Definitions

The following definitions are provided to facilitate an understanding of the present invention.

The singular forms “a,” “an,” and “the” include plural referents unless the context clearly dictates otherwise.

The term “isolated” is not meant to exclude artificial or synthetic mixtures with other compounds or materials, or the presence of impurities that do not interfere with the fundamental activity, and that may be present, for example, due to incomplete purification, or the addition of stabilizers.

The term “substantially pure” refers to a preparation comprising at least 50-60% by weight of a given material (e.g., nucleic acid, oligonucleotide, protein, etc.). Particularly, the preparation comprises at least 75% by weight, at least 80% by weight, at least 90% by weight, or at least 95% or more by weight of the given compound. Purity may be measured by methods appropriate for the given compound (e.g. chromatographic methods, agarose or polyacrylamide gel electrophoresis, HPLC analysis, and the like).

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Tween™ 80, polysorbate 80), emulsifier, buffer (e.g., Tris HCl, acetate, phosphate), antimicrobial, bulking substance (e.g., lactose, mannitol), excipient, auxiliary agent or vehicle with which an active agent of the present invention is administered. Pharmaceutically acceptable carriers can be sterile liquids, such as water and oils, including those of petroleum, animal, vegetable or synthetic origin. Water or aqueous saline solutions and aqueous dextrose and glycerol solutions are preferably employed as carriers, particularly for injectable solutions. Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, 20th Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3^{rd} Ed.), American Pharmaceutical Association, Washington, 1999.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition (e.g., microbial (e.g., bacterial) infection) resulting in a decrease in the probability that the subject will develop the condition.

A “therapeutically effective amount” of a compound or a pharmaceutical composition refers to an amount effective to prevent, inhibit, treat, and/or lessen the symptoms of a particular disorder or disease. The treatment of a microbial infection (e.g., a bacterial infection such as a S. aureus infection) herein may refer to an amount sufficient to inhibit microbial growth or kill the microbe and/or curing, relieving, and/or preventing the microbial infection, the symptom of it, or the predisposition towards it.

As used herein, the term “antibiotic” refers to antimicrobial agents for use in mammalian, particularly human, therapy. Antibiotics include, without limitation, beta-lactams (e.g., penicillin, ampicillin, oxacillin, cloxacillin, methicillin, and cephalosporin), carbacephems, cephamycins, carbapenems, monobactams, aminoglycosides (e.g., gentamycin, tobramycin), glycopeptides (e.g., vancomycin), quinolones (e.g., ciprofloxacin), moenomycin, tetracyclines, macrolides (e.g., erythromycin), fluoroquinolones, oxazolidinones (e.g., linezolid), lipopetides (e.g., daptomycin), aminocoumarin (e.g., novobiocin), co-trimoxazole (e.g., trimethoprim and sulfamethoxazole), lincosamides (e.g., clindamycin and lincomycin), polypeptides (e.g., colistin), and derivatives thereof.

As used herein, the term “subject” refers to an animal, particularly a mammal, particularly a human.

The term “promoter” as used herein refers to a DNA sequence which directs transcription of a polynucleotide sequence operatively linked thereto (e.g., in a cell). The promoter may also comprise enhancer elements which stimulate transcription from the linked promoter. The term “enhancer” refers to a DNA sequence which binds to the transcription initiation complex and facilitates the initiation of transcription at the associated promoter.

A “vector” is a nucleic acid molecule such as a plasmid, cosmid, bacmid, phage, or virus, to which another genetic sequence or element (either DNA or RNA) may be attached/inserted so as to bring about the replication and/or expression of the sequence or element (e.g., under the control of a promoter contained within the vector).

“Nucleic acid” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. An isolated nucleic acid (either DNA or RNA) may further represent a molecule produced directly by biological or synthetic means and separated from other components present during its production.

“Linker” refers to a chemical moiety comprising a covalent bond or a chain of atoms that covalently attach at least two compounds. The linker can be linked to any synthetically feasible position of the compounds, but preferably in such a manner as to avoid blocking the compounds desired activity. Linkers are generally known in the art. In a particular embodiment, the linker may contain from 0 (i.e., a bond) to about 50 atoms, from 0 to about 10 atoms, or from about 1 to about 5 atoms.

The term “antimicrobials” as used herein indicates a substance that kills or inhibits the growth of microorganisms such as bacteria, fungi, viruses, or protozoans, particularly bacteria.

The following example describes illustrative methods of practicing the instant invention and is not intended to limit the scope of the invention in any way.

### EXAMPLE

### Materials and Methods

### Peptide and Chemicals

Peptides were synthesized by the established solid-phase method and purified to >95% (Genemed Synthesis, TX). The quality of each peptide was determined based on Mass Spectrometry and HPLC. Peptides stock solutions were made by solubilizing in autoclaved distilled water and their concentrations were quantitated using UV spectroscopy based on the tryptophan (W) at 280 nm. For LL-37 and KR12, which do not contain a W, they were quantified by using the Waddell method (Waddell, W. J. (1956) J. Lab Clin Med., 48:311-4). Other chemicals were purchased from Sigma (MO) unless specified.

### Antibacterial Assays

The antibacterial activity of peptides was evaluated using a standard broth microdilution protocol (Clinical Laboratories Standards Institute (CLSI): M07-A10. Methods for Dilution Antimicrobial Susceptibility Tests for Bacteria that grow aerobically; Approved Standard, Tenth Edition, 2015) with minor modifications as described (Mishra, et al. (2019) Proc. Natl. Acad. Sci., 116:13517-13522). In brief, peptides (10 μL per well) with 2-fold dilutions were made in 96-well polystyrene microplates. Subsequently, the logarithmic phase bacterial cultures (i.e., optical density at 600 nm ˜0.5) were diluted to 0.001 OD and aliquoted to 90 μL per well. The plates were incubated overnight at 37°° C. Bacterial controls were treated with water, and the wells containing only the TSB media were used as blank. Positive controls included the parent peptide LL-37 as well as KR12. Post incubation, the plates were read at 630 nm using ChroMate® 4300 Microplate Reader (Awareness Technology, FL). The minimal inhibitory concentration (MIC) was the lowest peptide concentration that fully inhibited bacterial growth.

To study the influence of medium conditions on the antimicrobial activity of peptides against S. aureus USA300, different pH, sodium chloride (NaCl), human sera were included into the antibacterial assays. Control experiments in TSB without any additions (pH 7.4) were set up in the same manner. In addition, antimicrobial activities of human LL-37, KR12, and C10-KR8d were compared in 10%, 50%, and 100% TSB as well as in TSB and MHB using E. coli ATCC 25922 and S. aureus USA300 LAC.

### Orientational Immobilization of (10-KR8 Peptides on M370 Beads

Dynabeads M-370 amine (2×10⁹ beads/mL, 50 μL; Thermo Fisher Scientific) were washed 3 times in sterile PBS (1 mL; GIBCO) and reacted with a bifunctional SM(PEG)₄ cross-linker (5 mg/mL in DMSO) containing N-hydroxysuccinimide (NHS) ester and maleimide functional groups. A stable covalent bond between the amino group of beads and the NHS ester of the cross-linker was formed in PBS at pH 7.2 at room temperature for 24 hours with shaking at 80 rpm. The beads were then washed six times with PBS to remove nonreacted cross-linker. Next, peptide C10-KR8 (with a cysteine at the N-terminus) at 5 mg/mL was reacted with the free maleimide group in PBS for 24 hours at room temperature with shaking at 80 rpm. Another round of washing was conducted to remove the nonreacted peptides. Both the L-and D-forms of the peptide were coupled in the same manner. A portion of the beads without peptide coupling was saved as a control.

For serum binding experiments, the beads were resuspended in 500 μL of PBS; 200-μL aliquots of the coated and control dynabead were incubated with 5% mouse plasma for 18 hours at 37° C. After binding, the beads were washed six times with PBS to remove unbound proteins. Finally, the beads were suspended in 50 μL of PBS for mass spectrophotometric analysis to identify peptide-binding partners.

### Mass Spectrometric Identification of Serum Proteins Bound to Peptides

The samples were reduced by 10 mM dithiothreitol (DTT) and incubated for 30 minutes at 56° C. and back to room temperature. A final concentration of 50 mM iodoacetamide was added to alkylate free cysteines followed by further incubation for 20 min in the dark. Bound proteins were enzymatically digested by trypsin (4 μL of 0.5 μg/mL) overnight. A 60 ∥L portion was transferred to a new Eppendorf® tube and dried for 2 hours. The samples were then dissolved in 20 μL of 2% acetonitrile with 0.1% formic acid before being loaded to Q-tip columns (Pepclean, C18 spin column). Column was activated by pipetting in and out (4× times), 10 μL of 70% acetonitrile with 0.1% formic acid, and equilibrating with the same volume of 2% acetonitrile with 0.1% formic acid. Binding of the samples was done by mixing more than 10 × times, followed by washing in the same buffer (2% acetonitrile with 0.1% formic acid). The bound peptides were then eluted with 20 μL of 70% acetonitrile with 0.1% formic acid. Samples were subjected to LC-MS analysis after being suspended in the desired buffer.

Extracted peptides were resuspended in 2% acetonitrile (ACN) and 0.1% formic acid (FA) and loaded onto trap column Acclaim™ PepMap™ 100 75 μm×2 cm C18 LC Columns (Thermo Scientific) at a flow rate of 4 μL/minute. These were then separated with a Thermo RSLC UltiMate™ 3000 on a Thermo Easy-Spray PepMap™ M RSLC C18 75 μm×50 cm C-18 2 μm column (Thermo Scientific) with a step gradient of 4-25% solvent B (0.1% FA in 80% ACN) from 10 to 37 minutes and 25-45% solvent B for 37-46 minutes at 300 nL/min and 50° C. with a 70 minute total run time. Eluted peptides were analyzed by a Thermo Orbitrap Fusion™ Lumos™ Tribrid™ (Thermo Scientific) mass spectrometer in a data dependent acquisition mode. A survey full scan MS (from m/z 350-1800) was acquired in the orbitrap with a resolution of 120000. The AGC target for MS1 was set as 4×10⁵ and the ion filling time set as 100 ms. The most intense ions with charge state 2-6 were isolated in 3 second cycle and fragmented using HCD fragmentation with 40% normalized collision energy and detected at a mass resolution of 30000 at 200 m/z. The AGC target for MS/MS was set as 5×10⁴ and ion filling time set 60 ms dynamic exclusion was set for 30 seconds with a 10 ppm mass window. Protein identification was performed by searching MS/MS data against the Swiss-Prot mouse protein database. The search was set up for full tryptic peptides with a maximum of two missed cleavage sites. Acetylation of protein N-terminus and oxidized methionine were included as variable modifications, and carbamidomethylation of cysteine was set as fixed modification. The precursor mass tolerance threshold was set at 10 ppm, and maximum fragment mass error was 0.02 Da. Qualitative analysis was performed using PEAKS 8.5 software. The significance threshold of the ion score was calculated based on a false discovery rate of ≤1%.

### Protease Stability of the Designed Peptides

The peptide was incubated with various proteases in 10 mM PBS buffer (pH 7.4) at 37° C. for 24 hours. Aliquots (20 μL) of the reaction solutions were taken at 3 and 24 hours. The reaction was then stopped by mixing with 20 μL of 2×SDS loading buffer and boiling in a water bath for ˜10 minutes. For the SDS-PAGE analysis, 10 μL of each sample was loaded to the well of a 5% stacking/18% resolving tricine gel and run at a constant current of 35 mA. The gels were stained using Coomassie Brilliant Blue.

### Bacterial Membrane Permeation

The experiment was performed as described with minor modifications (Mishra, et al. (2019) Proc. Natl. Acad. Sci., 116:13517-13522). Serially diluted 10×peptides (10 μL each well) were created in 96-well microtiter plates. Propidium iodide (2 μL) at a fixed concentration of 20 μM was added to each well followed by 88 μL of the exponential phase S. aureus USA300 culture (a final OD600˜0.1 in TSB media or PBS). Both daptomycin (98%, TSZChem, MA) and rifamycin (98%, Alfa Aesar, MA) were used as controls. The plate was incubated at 37° C. with continuous shaking at 100 rpm in a FLUOstar® Omega (BMG LABTECH, NC) microplate reader. The samples fluorescence was read at every 5 minutes for 24 cycles with excitation and emission wavelengths of 584 and 620 nm, respectively. Plots were generated using average values from the experiments using GraphPad Prism 7.

### Bacterial Membrane Depolarization

The experiment was conducted as described (Marks, et al. (2013) PLOS One 8:e63158). In brief, an overnight culture of S. aureus USA300 was subcultured in a fresh TSB medium and grown to the exponential phase. Cells were spun using centrifugation, washed 2× with PBS, resuspended in twice the volume of PBS containing 25 mM glucose, and incubated at 37° C. for 15 minute. For membrane depolarization measurements, 500 nM (final concentration) of the dye DiBAC4 (3) bis(1,3-dibutylbarbituric acid) trimethine oxonol (ANASPEC, CA) was added, and the reaction mixture was vortexed gently. Aliquots of 90 μL of the energized bacteria solution were loaded to the wells, and the plate was fed into a FLUOstar® Omega microplate reader. Fluorescence was read for 20 minutes at excitation and emission wavelengths of 485 and 520 nm, respectively, to get dye normalization. Then, 10 μL of the peptide solution was added and gently mixed. Fluorescence readings were recorded for 40 minutes, where Triton™ X-100 (0.1%) was used as a positive control. Also, both daptomycin and 17BIPHE2 (Wang, et al. (2014) ACS Chem. Biol., 9:1997-2002) were included for comparison.

### Circular Dichroism (CD)

CD spectra were measured on a Jasco J-815 spectropolariometer at UNMC in the far-UV region from 260-190 nm with a 1 nm interval, a 2 nm bandwidth using a digital integration time of 4 seconds, and a scan speed of 20 nm per minute. During measurements, the high-tension signal applied to the detector was also recorded and was subsequently converted to absorbance. Each spectrum represented the average of five individual scans with a corresponding reference measurement on pure solvent subtracted. The peptide (C10-KR8 or C10-KR8d) concentration was fixed at 1 mM in 10 mM PBS (pH 7) or in the presence of 60 fold SDS (molar ratio). Each sample was placed in a 0.1 mm quartz cuvette. The temperature was kept at 25° C. during measurements. Data were processed and converted to molar ellipticity ([θ]) using the Jasco Spectra Analysis software and plotted using GraphPad Prism 7.

### Bacterial Resistance Development to the Peptide

This experiment was performed similar to the MIC determination with a few modifications as described (Mishra, et al. (2019) Proc. Natl. Acad. Sci., 116:13517-13522). In short, an exponential phase S. aureus USA300 culture (i.e., optical density at 600 nm≈0.5) was diluted and partitioned into a 96-well polystyrene microplate with ˜10⁵ bacteria per well (90 μL aliquots). After treatment with 10 μL of peptide or nafcillin solutions at various concentrations, microplates were incubated at 37° C. overnight and read on a ChroMate® 4300 Microplate Reader at 630 nm (Awareness Technology, FL). Of the wells with sub-MIC levels of the peptides that retained growth, approximately half of the control wells were again reinoculated in fresh TSB with sub-MIC concertation of peptides or antibiotics to attain exponential phase for MIC determination. Up to 15 serial passages of the bacteria cultures were conducted. The increase in the fold change (MIC on given passage/MIC recorded in first day of passage) was used to determine the degree of drug resistance.

Antimicrobial Susceptibility of LL-37 Susceptible S. aureus Strains to (10-KR8d

The antimicrobial susceptibility of over 20 MRSA transposon mutants were also tested, based on a transposon library screening, in the presence of LL-37 peptides (Golla, et al. (2020) ACS Infect. Dis., 6:1866-1881). This Nebraska Transposon Mutant Library of S. aureus USA300 consists of 1920 mutants, each with one nonessential gene disabled due to a transposon insertion (Fey, et al. (2013) mBio 4:e00537-12). The correct insertion of the transposon to each Staph mutant had been verified (Golla, et al. (2020) ACS Infect. Dis., 6:1866-1881). Antimicrobial susceptibility to the new peptide C10-KR8d was evaluated in the same manner as MIC assays. Susceptible strains could be inhibited at a peptide concentration lower than the MIC against the wild strain S. aureus JE2.

### Hemolytic Assays

Hemolytic assays of peptides were performed as described (Lakshmaiah, et al. (2020) Proc. Natl. Acad. Sci., 117:19446-19454). Briefly, human red blood cells (hRBCs) were obtained from UNMC Blood Bank. The cells were washed 3× with isotonic saline (0.9% NaCl) and diluted to 2% (v/v). Peptides at various concentrations were added to the 2% blood cells and incubated at 37° C. for 1 hour. Post-incubation, the plates were spun at 2000 rpm for 10 minutes; aliquots of the supernatant were carefully transferred to a fresh 96-well microplate. The amount of cells lysed is proportional to the hemoglobin released and was measured at 545 nm using a ChroMate® microplate reader (Awareness Technology, FL). The percent lysis was calculated by assuming 100% release when human blood cells were treated with 1% Triton™ X-100, and 0% release when incubated with PBS. The peptide concentration that caused 50% lysis of hRBCs is defined as HC₅₀. The cell selectivity index (CSI) was calculated as the ratio between HC₅₀ and the MIC of the corresponding peptide against MRSA.

### Mammalian Cell Viability Assay

Peptides were assessed for potential cytotoxicity using HaCaT cells (Addexbio Technologies, CA). Briefly, cells were seeded at a density of 1×10⁴ per well in a tissue culture 96-well plate. DMEM was supplemented with 10% fetal bovine serum (FBS) and incubated at 37° C. in a 5% CO₂ atmosphere for 24 hours. Culture medium was aspirated and replaced with fresh serum free media. Cells were exposed to different concentrations of peptides for 1 hour at 37° C. in a 5% CO₂ atmosphere. Post-incubation, the cells were washed and incubated with 100 μL of DMEM media containing 20 μL of MTS reagent for 2 hours at 37° C. Absorbance was read at 492 nm using a ChroMate® microplate reader (Awareness Technology, FL).

### In Vivo Toxicity

Toxicity was assessed in female C57BL/6 mice by the intraperitoneal administration of increasing doses of the antimicrobial peptide C10-KR8d (10, 20, and 40 mg/kg) and a control group without peptide treatment. The animals were then observed for 5 days (twice a day). The number of moribund/dead animals for each of the doses was noted and plotted.

Mice were caged, fed, and environmentally adapted as described (Lakshmaia, et al. (2020) Proc. Natl. Acad. Sci., 117:19446-19454). All animal manipulations were performed in a class II laminar flow biological safety cabinet.

### In Vivo Efficacy of Peptides in Neutropenic Murine Infection Model

Female C57BL/6 mice (6 weeks old) were purchased from Charles River. After environmental adaptation, mice were induced to be neutropenic by the administration of two doses of cyclophosphamide on Day 1 (150 mg/kg) and Day 4 (100 mg/kg). On Day 5, mice were infected with S. aureus USA300 (˜2×10⁶ CFU per mouse) via intraperitoneal injection (i.p.) (Lakshmaia, et al. (2020) Proc. Natl. Acad. Sci., 117:19446-19454). For the treatment groups, mice were i.p. treated 2 hours post infection with peptides at a single dose of 5 mg/kg. This bacterial strain was able to disseminate to different organs 2 hours post infection (Lakshmaia, et al. (2020) Proc. Natl. Acad. Sci., 117:19446-19454). At the end of the experiments, all the animals were sacrificed according to institutional guidelines. Organs, including spleen, liver, lung, and kidney, were harvested, weighed, and placed in 1 mL of sterile PBS and stored on ice. Harvested organs were subsequently homogenized using an Omni Homogenizer. Proper dilutions were made to get countable colonies. The homogenates were plated onto blood agar plates and incubated overnight at 37° C. The CFU of each murine tissue was plotted as an individual point, and error bars represent the deviation within the experimental group. * indicates p<0.05, **p<0.01, ***p<0.001, and NS means no significance (determined by Mann-Whitney test).

### Catheter-Associated Murine Biofilm Model

Peptide potency was evaluated in the catheter model as described (Wang, et al. (2014) ACS Chem. Biol., 9:1997-2002; Heim, et al. (2014) Methods Mol. Biol., 1106:183-191). On the day of infection, mice were anesthetized using ketamine/xylazine (100/10 mg/kg). A small incision was made on the left flank region of the mouse, and using small blunt spatula, a pouch was made for catheter insertion. A sterile catheter with a length of 1 cm was inserted into the pouch aseptically. The wound was sealed using wound closure VetBond™ glue. Bacterial inoculum (20 μL with 10³ CFU) was injected into the lumen of the catheter. Two hours post infection, the peptide was injected at 15 mg/kg (50 μL at each of the five sites in and around the catheter). Mice were allowed full recovery from anesthesia in an oxygen-enriched chamber. Three days after infection, untreated and treated animal groups were CO₂ euthanized and the tissue around the catheter was harvested along with the catheter. A bacterial uninfected catheter group was included as a negative control. All the catheters were sonicated at 37 kHz for 15 minutes to release the bacteria in biofilms, and the tissues were homogenized. Appropriate dilutions of tissue homogenates were made, plated onto the blood agar, and incubated at 37° C. overnight. The CFU of each mouse was plotted as an individual point, and error bars represent the deviation within the experimental group. Degree of significance was represented as *p<0.05**p<0.01, ***p<0.001, and NS means no significance (determined by nonparametric, unpaired, Mann-Whitney test).

### Cytokine Quantification by Flow Cytometry

The catheter-associated tissue homogenates/plasma samples stored at −80° C. were thawed to room temperature for the cytokines quantification using the BioLegend® individual mouse cytokine kit. The protocol for sample preparation and analysis was per the manufacturer manual. The samples were in duplicates with appropriate standards. The data are represented as mean±SD. Plots were generated using GraphPad prism 7, where * indicates p<0.05, **p<0.01, ***p<0.001, and NS means no significance (nonparametric, unpaired, Mann-Whitney test).

### Results

### Systematic Search of Selective Peptides by a 2D Molecular Array

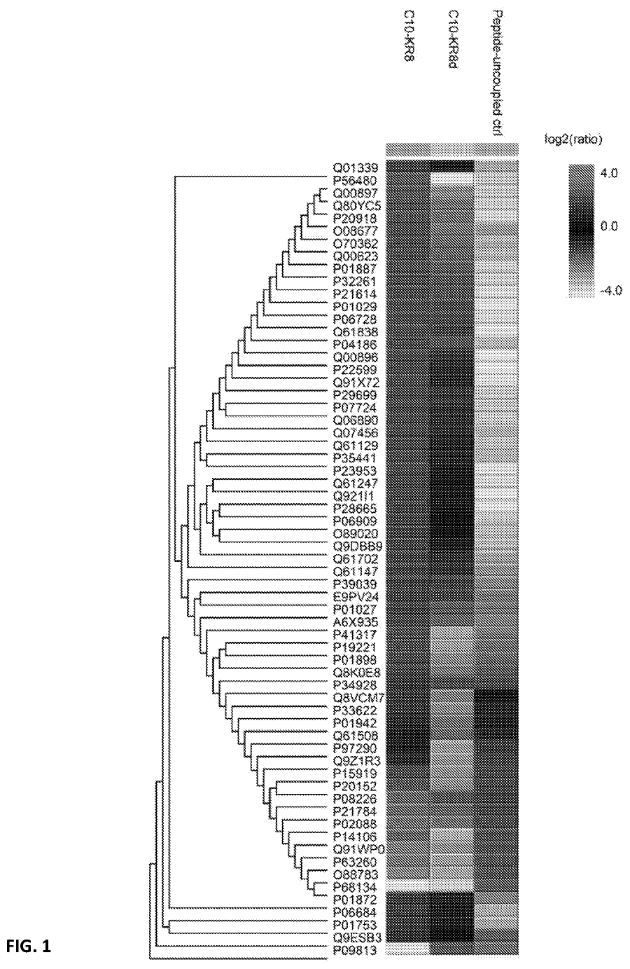

To identify highly selective antimicrobial peptides, the peptide length from 4 to 12 amino acids (aa) and the fatty acid chain length from C6 to C14 were varied with a step size of 2 in both cases. Various peptide segments were generated by truncating the KR12 peptide sequence (KRIVQRIKDFLR (SEQ ID NO: 7)-amide) from the C-terminus, two residues at a time (Wang, G. (2008) J. Biol. Chem., 283:32637-32643). In each case, a fatty acid was attached to the N-terminal amine of the peptide segment by forming an amide bond. In addition, the underlined valine was replaced by a tryptophan (W) residue to facilitate peptide quantification by UV spectroscopy at 280 nm. The generated peptides were named according to the nomenclature convention for LL-37. The names of these lipopeptides are represented using a general format Cm-KRn, where Cm stands for a fatty acid with m carbons, whereas KRn means a peptide with n amino acids and starting with KR (n is an even number here). As controls, both human LL-37 and its smallest antibacterial peptide KR12 were included. Antimicrobial activities of the peptides were tested using a panel of bacteria, including E. coli, K. pneumoniae, P. aeruginosa, and S. aureus. The minimal inhibitory concentrations (MIC) of this library of peptides are listed in Table 1. LL-37 was not active against S. aureus USA300 in 100% trypic soy broth (TSB), but inhibited Gram-negative E. coli ATCC 25922 (MIC 3.1 μM). Under such a condition, KR12 was not active against MRSA and weakly active against E. coli (MIC 50 μM). These results agree with the previous MIC data (Epand, et al. (2009) Antimicrob. Agents Chemother., 53:3705-3714). However, most of these KR12 derived peptides gained activity against MRSA after attaching an acyl chain at the N-terminus. Table 1 shows that those with very short peptide segments or fatty acid chains were inactive against S. aureus. The active peptides contained C8 to C14 fatty acid chains with MIC in the range of 1.5-12.5 μM. Peptide toxicity was also evaluated by measuring the ability of the peptide to lyse human red blood cells. The concentration that causes 50% hemolysis (HC₅₀ in Table 1) was estimated for each peptide based on peptide dose-dependent lysis data. Some peptides conjugated with a C6 (aa4-aa10), C8 (aa4-aa8), or C10 (aa6 and aa8) fatty acid chain showed poor hemolytic activity (HC₅₀>200 μM in Table 1). These results rejected both the C12 (HC₅₀<75 μM) and C14 (HC₅₀<25 μM) fatty acid chains (FIG. 1B) due to higher hemolysis. They also allowed the rejection of the peptides with a C6 fatty acid chain owing to either high hemolytic ability or weak antibacterial activity against all the bacteria tested in Table 1. Two peptides, C10-KR8 and C8-KR10, were found to be less hemolytic. Both are active against S. aureus, E. coli, and P. aeruginosa (MIC 1.6-12.5 μM). They also inhibited clinical strains of S. aureus, including USA200, USA400, Newman, and Mu50 (1.6-6.2 μM in Table 2). However, based on cell selectivity index (CSI), i.e., the ratio of HC₅₀ and MIC (Table 1), C10-KR8 (CSI 200) was more selective than C8-KR10 (CSI 40-80). Therefore, C10-KR8 is used for detailed study below. Its L-form (made of L-amino acids) and D-form (made of D-amino acids) have the same CSI in Table 1. The D-form is shortened into C10-KR8d.

| | | | MIC |
|---|---|---|---|
| | | Acyl | (μM) |
| Peptide | Amino Acid Sequence | Chain | EC |
| LL-37 | LLGDFFRKSKEKIGKEFK | None | 3.1-6.2 |
| | RIVQRIKD-FLRNLVPRT | | |
| | ES (SEQ ID NO: 1) | | |
| KR12 | KRIWQRIKDFLR | None | >50 |
| | (SEQ ID NO: 2) | | |
| C14-KR10 | KRIWQRIKDF (SEQ ID NO: 3) | C14 | 50 |
| C14-KR8 | KRIWQRIK (SEQ ID NO: 4) | C14 | 12.5 |
| C14-KR6 | KRIWQR (SEQ ID NO: 5) | C14 | 12.5 |
| C14-KR4 | KRIW (SEQ ID NO: 6) | C14 | 12.5-50 |
| C12-KR12 | KRIWQRIKDFLR | C12 | >50 |
| | (SEQ ID NO: 2) | | |
| C12-KR10 | KRIWQRIKDF (SEQ ID NO: 3) | C12 | 6.2 |
| C12-KR8 | KRIWQRIK (SEQ ID NO: 4) | C12 | 6.2 |
| C12-KR6 | KRIWQR (SEQ ID NO: 5) | C12 | 12.5 |
| C12-KR4 | KRIW (SEQ ID NO: 6) | C12 | 12.5 |
| C10-KR12 | KRIWQRIKDFLR | C10 | >50 |
| | (SEQ ID NO: 2) | | |
| C10-KR10 | KRIWQRIKDF (SEQ ID NO: 3) | C10 | 3.1 |
| C10-KR8 | KRIWQRIK (SEQ ID NO: 4) | C10 | 12.5 |
| C10-KR8d | KRIWQRIK (SEQ ID NO: 4) | C10 | 12.5 |
| C10-KR6 | KRIWQR (SEQ ID NO: 5) | C10 | 50 |
| C10-KR4 | KRIW (SEQ ID NO: 6) | C10 | 50 |
| C8-KR12 | KRIWQRIKDFLR | C8 | 6.2 |
| | (SEQ ID NO: 2) | | |
| C8-KR10 | KRIWQRIKDF (SEQ ID NO: 3) | C8 | 6.2 |
| C8-KR10d | KRIWQRIKDF (SEQ ID NO: 3) | C8 | 6.2 |
| C8-KR8 | KRIWQRIK (SEQ ID NO: 4) | C8 | 25 |
| C8-KR6 | KRIWQR (SEQ ID NO: 5) | C8 | 50 |
| C8-KR4 | KRIW (SEQ ID NO: 6) | C8 | >50 |
| C6-KR12 | KRIWQRIKDFLR | C6 | 3.1 |
| | (SEQ ID NO: 2) | | |
| C6-KR10 | KRIWQRIKDF (SEQ ID NO: 3) | C6 | 50 |
| C6-KR8 | KRIWQRIK (SEQ ID NO: 4) | C6 | >50 |
| C6-KR6 | KRIWQR (SEQ ID NO: 5) | C6 | >50 |
| C6-KR4 | KRIW (SEQ ID NO: 6) | C6 | >50 |

TABLE 1
Antimicrobial and Hemolytic Activities of KR12 Derived
Lipopeptides. All peptides are C-terminally amidated.

| | MIC | MIC | MIC | | |
|---|---|---|---|---|---|
| | (μM) | (μM) | (μM) | HC₅₀ | |
| Peptide | KP | PA | SA | (μM) | CSI |
| LL-37 | 6.2 | >50 | >50 | 170 | ND |
| KR12 | >50 | 25 | >50 | >340 | ND |
| C14-KR10 | 6.2 | 12.5 | 3.1 | <25 | <8 |
| C14-KR8 | >50 | 6.2 | <1.5 | <25 | <16 |
| C14-KR6 | >50 | 6.2 | 3.1 | <25 | <8 |
| C14-KR4 | 12.5 | 6.2 | 3.1 | <25 | <8 |
| C12-KR12 | 12.5 | >50 | 6.2 | <25 | <4 |
| C12-KR10 | 3.1 | 3.1 | 1.5 | <25 | <16 |
| C12-KR8 | >50 | 3.1 | 1.5 | 35 | 24 |
| C12-KR6 | >50 | 6.2 | 3.1 | 75 | 24 |
| C12-KR4 | >50 | 6.2 | 3.1 | 30 | 10 |
| C10-KR12 | 6.2-12.5 | 25 | 3.1 | <25 | <8 |
| C10-KR10 | 6.2 | 6.2 | 1.5 | 50 | 33 |
| C10-KR8 | 50 | 6.2 | 1.5-3.1 | 300 | 200 |
| C10-KR8d | 25 | 6.2 | 1.5-3.1 | 300 | 200 |
| C10-KR6 | >50 | 25 | 12.5 | >340 | >27 |
| C10-KR4 | >50 | 12.5 | 12.5 | 150 | 12 |
| C8-KR12 | <6.2 | 6.2 | 3.1 | <25 | <8 |
| C8-KR10 | 25 | 6.2 | 3.1-6.2 | 250 | 40-80 |
| C8-KR10d | 12.5 | 12.5 | 3.1 | 150 | 48 |
| C8-KR8 | >50 | 12.5 | 6.2 | >200 | 32 |
| C8-KR6 | >50 | >50 | >50 | >200 | ND |
| C8-KR4 | >50 | >50 | >50 | >200 | ND |
| C6-KR12 | 6.2 | 12.5 | <1.5 | 25 | 16 |
| C6-KR10 | >50 | 50 | 25 | >200 | >8 |
| C6-KR8 | >50 | >50 | >50 | >200 | ND |
| C6-KR6 | >50 | >50 | >50 | >200 | ND |
| C6-KR4 | >50 | >50 | >50 | >200 | ND |

MIC, the minimal inhibitory concentration; EC, E. coli ATCC 25922; KP, Klebsiella pneumonia ATCC 13883; PA, P. aeruginosa PAO1; SA, S. aureus USA300 LAC. ND = not determined. CSI = HC₅₀/MIC_{SA} where HC₅₀ is the peptide concentration that causes 50% hemolysis and MIC_{SA} is MIC against S. aureus. MIC and HC₅₀ of LL-37 were taken from Narayana, et al. (Biochim. Biophys. Acta, Biomembr. (2019) 1861: 1592-1602).

TABLE 2
Minimal inhibitory concentrations (μM) of LL-37
lipopeptides against S. aureus clinical strains.

| S. aureus strain | USA200 | USA400 | Newman | Mu50 |
|---|---|---|---|---|
| C10-KR8 | 3.1 | 3.1 | 3.1 | 3.1 |
| C10-KR8d | 1.6 | 1.6 | 1.6 | 3.1 |
| C8-KR10 | 6.2 | 3.1 | 6.2 | 6.2 |
| C8-KR10d | 3.1 | 3.1 | 3.1 | 3.1 |

### Antimicrobial Robustness of the Selective Lipopeptides

In addition to a long peptide length, another weakness of LL-37 is its loss of antimicrobial activity under certain conditions (Wang, et al. (1998) J. Biol. Chem., 273:33115-8; Wang, et al. (2004) Rapid Commun. Mass Spectrom., 18:588; Mishra, et al. (2017) Curr. Opin. Chem. Biol., 38:87-96). Antimicrobial activity of LL-37, KR12, and C10-KR8d was compared in different media: Mueller Hinton Broth (MHB) and TSB. These peptides showed essentially the same MIC values (Table 3). The MICs were also evaluated in media containing varying amounts of TSB. In 50% or 100% TSB, both LL-37 and KR12 were not active against MRSA up to 50 μM, but started to gain activity at 10% TSB. In the case of E. coli ATCC 25922, both LL-37 and KR12 showed an inhibitory effect, although KR12 was less active in 100% TSB (Table 3). It appears that the anti-MRSA activity of both LL-37 and KR12 were more influenced by the concentration of TSB. However, the newly identified peptide C10-KR8d retained antibacterial activity against both MRSA and E. coli under various TSB concentrations as well as in different media. To corroborate peptide robustness, antimicrobial activity of both the L-and D-forms of C10-KR8 was tested in the presence or absence of physiological salts and at different pH values. Notably, sodium chloride did not alter the MIC values of these selective lipopeptide peptides at 100 or 200 mM (Table 4). Likewise, these peptides showed nearly the same activity at different pH conditions, from 6.8 to 8 (Table 4). However, human serum had a definitive effect on anti-MRSA activity depending on the chirality of the peptide. While the L-form rapidly lost activity in the presence of 5 or 10% human serum (MIC >25 M), the activity of the D-form of C10-KR8 was only slightly reduced by 2-fold at 10% serum, pH 7.4. To validate the finding with C10-KR8, the antibacterial activity of the L-and D-forms of C8-KR10 (octanoic acid+KRIWQRIKDF (SEQ ID NO: 3)-amide) under these conditions was also compared. The same results were obtained, i.e., the D-form of C8-KR10 (C8-KR10d) is more robust than the L-form (Table 4). These D-form peptides are less likely to bind to serum proteins.

TABLE 3
Effects of TSB Medium Percentages and Different Media on the
Antibacterial Activity of C10-KR8d, KR12, and Human LL-37.

| | | | | |
|---|---|---|---|---|
| Peptide | 10% TSB | 50% TSB | 100% TSB | 100% MHB |

Minimal inhibitory concentration (μM) against S. aureus

| | | | | |
|---|---|---|---|---|
| LL-37 | 50 | >50 | >50 | >50 |
| KR12 | 6.2 | >50 | >50 | >50 |
| C10-KR8d | 1.6 | 3.1 | 3.1 | 3.1 |

Minimal inhibitory concentration (μM) against E. coli

| | | | | |
|---|---|---|---|---|
| LL-37 | 1.6 | 6.2 | 6.25-12.5 | 6.2 |
| KR12 | 1.6 | 3.1 | >50 | >50 |
| C10-KR8d | 1.6 | 3.1 | 6.2 | 3.1 |

TABLE 4
Effects of Salts, pH, and Human Serum on Peptide Activity
against Staphylococcus aureus USA300 LAC. The MIC
was normally obtained in 100% TSB at pH 7.4 (indicated
by 0 mM in the table). The condition of the TSB was then
altered by adjusting pH, adding NaCl or human serum.

| | Minimal inhibitory concentration (MIC, μM) | | |
|---|---|---|---|
| Peptide | 0 mM | 100 mM | 200 mM |
| C10-KR8 | 3.1 | 3.1 | 3.1 |
| C10-KR8d | 3.1 | 3.1 | 3.1 |
| C8-KR10 | 6.2 | 6.2 | 6.2 |
| C8-KR10d | 3.1 | 3.1 | 3.1 |

| | Minimal inhibitory concentration (MIC, μM) | | | | |
|---|---|---|---|---|---|
| Peptide | pH = 6.8 | pH = 7.4 | pH = 8.0 | 5% serum | 10% serum |
| C10-KR8 | 3.1-6.2 | 3.1 | 1.6 | >25 | >25 |
| C10-KR8d | 3.1 | 3.1 | 1.6 | 3.1-6.2 | 6.2 |
| C8-KR10 | 12.5 | 6.2 | 3.1 | >25 | >25 |
| C8-KR10d | 6.2 | 3.1 | 1.6 | 3.1 | 6.2 |

### Binding to Serum Proteins

The loss of peptide activity in the presence of serum might result from binding to serum proteins. To provide evidence, a peptide binding study was conducted using both the L-and D-forms of C10-KR8. In this experiment, the peptide was attached to a commercial bead via two steps of chemical reactions. The peptide immobilization to the beads allowed murine serum proteins to bind to the peptide and facilitated a thorough wash to remove unbound molecules. The bound proteins were identified by a proteomic mass spectrometry (MS) study. The top-20 bound proteins are provided in a heat map in FIG. 1 based on protein relative abundance. These proteins appeared to preferentially associate with the L-form of C10-KR8. Note that 16 proteins were associated with the linker region of the beads without peptide coupling. Among them, five (Q8VCM7, P33622, P01942, Q61508, and P97290) overlapped with those detected in the presence of C10-KR8, but none of these 16 proteins were clearly associated with C10-KR8d. Thus, the type of “baits” on the beads determines the types of the bound proteins. Mouse plasma proteins that associate with bead-attached C10-KR8 peptides include UniProtKB/Swiss-Prot Accession ID Nos. P07724 (serum albumin), Q92111(serotransferrin), P01027 (complement C3), Q61838 (pregnancy zone protein), Q00623 (apolipoprotein A-I), P20918 (plasminogen), P28665 (murinoglobulin-1), E9PV24 (fibrinogen alpha chain), Q8KOE8 (fibrinogen beta chain), Q91X72 (Hemopexin), P23953 (carboxylesterase 1C), Q00896 (alpha-1-antitrypsin 1-3), Q8VCM7 (fibrinogen gamma chain), Q61147 (ceruloplasmin), P02088 (hemoglobin subunit beta-1), 008677 (kininogen-1), P19221 (prothrombin), P06684 (complement C5), P21614 (vitamin D-binding protein), P32261 (antithrombin-III), P06728 (apolipoprotein A-IV), P04186 (complement factor B), P01872 (immunoglobulin heavy constant mu), P35441 (thrombospondin-1), Q01339 (beta-2-glycoprotein 1), P29699 (alpha-2-HS-glycoprotein), Q61247 (alpha-2-antiplasmin), Q06890 (clusterin), Q9DBB9 (carboxypeptidase N), P15919 (V(D)J recombination-activating protein 1), Q07456 (protein AMBP), Q61702 (inter-alpha-trypsin inhibitor heavy chain), P14106 (complement C1q subcomponent subunit B), P06909 (complement factor H), O89020 (afamin), O70362 (phosphatidylinositolglycan-specific phospholipase D), A6X935 (inter alpha-trypsin inhibitor heavy chain 4), Q80YC5 (coagulation factor XII), P01898 (H-2 class I histocompatibility antigen Q10 alpha chain), P68134 (actin alpha skeletal muscle), P41317 (mannose-binding protein C), P08226 (apolipoprotein E), P01942 (hemoglobin subunit alpha), Q00897 (alpha-1-antitrypsin 1-4), Q9Z1R3 (apolipoprotein M), Q9ESB3 (histidine-rich glycoprotein), P34928 (apolipoprotein C-I), P63260 (actin cytoplasmic 2), P09813 (apolipoprotein A-II), P20152 (vimentin), P21784 (V(D)J recombination-activating protein 2), P56480 (ATP synthase subunit beta mitochondrial), Q61129 (complement factor I), P01753 (Ig heavy chain V region), P01029 (complement C4-B), P01887 (Beta-2-microglobulin), P22599 (alpha-1-antitrypsin 1-2), Q61508 (extracellular matrix protein 1), Q91WPO (mannan-binding lectin serine protease 2), 088783 (coagulation factor V), P97290 (plasma protease C1 inhibitor), P33622 (apolipoprotein C-III), and P39039 (mannose-binding protein A). Notably, the list includes proteins such as albumin, complement proteins, and numerous apolipoproteins. Table 5 calculated the intensity ratios of these apolipoproteins bound to the L-and D-forms of C10-KR8. It is evident that most of the apolipoproteins, including A-I, A-IV, C-I, and C-III, showed a preferred binding to the L-form of C10-KR8. Interestingly, both apoA-II and apoE were able to bind the linker without the peptide. It appeared that the D-form peptide could enhance the binding of apoA-II, whereas the apoE binding to the linker was slightly reduced in the presence of either the L-or D-form of the peptide. Therefore, unlike the D-form, the L-form of C10-KR8 is capable of binding to numerous serum proteins, including apolipoproteins.

TABLE 5
Intensity ratios of mouse apolipoproteins bound to the
L- and D-forms of C10-KR8 covalently attached to beads.

| | | C10- | C10- | | |
|---|---|---|---|---|---|
| Acces- | | KR8 + | KR8d + | | L/D |
| sion ID | Name | linker | linker | Linker | Ratio |
| P07724 | Serum albumin | 3.72E+10 | 1.63E+10 | 2.78E+09 | 2.28 |
| Q00623 | Apolipoprotein | 4.29E+09 | 1.21E+09 | 3.52E+08 | 3.54 |
| | A-I | | | | |
| P09813 | Apolipoprotein | 3.65E+05 | 2.49E+08 | 5.67E+07 | 0.00147 |
| | A-II | | | | |
| P06728 | Apolipoprotein | 3.21E+08 | 1.11E+08 | 1.44E+07 | 2.89 |
| | A-IV | | | | |
| P34928 | Apolipoprotein | 4.35E+08 | 2.09E+08 | 2.12E+08 | 2.08 |
| | C-I | | | | |
| P33622 | Apolipoprotein | 2.02E+08 | 5.57E+07 | 1.69E+08 | 3.62 |
| | C-III | | | | |
| P08226 | Apolipoprotein | 1.68E+07 | 2.07E+07 | 5.14E+07 | 0.811 |
| | E | | | | |

### Peptide Stability to Proteases

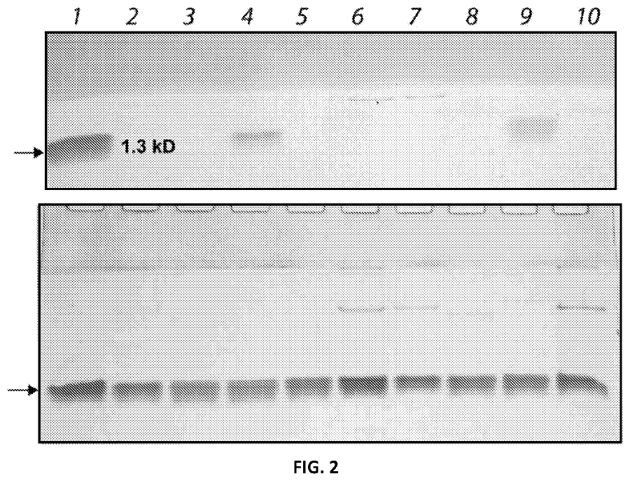

Protease degradation makes many peptides unavailable as oral drugs. Also, the cleavage of human LL-37 constitutes a virulence mechanism for bacterial infection (Sieprawska-Lupa, et al. (2004) Antimicrob. Agents Chemother., 48:4673-9). The loss of peptide activity in the antimicrobial assays in the presence of human serum could result from both protein binding and protease cleavage. The serum protein binding above validated the preferred binding of the L-form than the D-form to serum proteins. To illustrate that protease cleavage also plays a role, peptide levels were compared in the presence of five known proteases. The major antimicrobial region of LL-37 is degraded in vitro in 4 hours (Wang, et al. (2014) ACS Chem. Biol., 9:1997-2002). In line with this, the L-form of C10-KR8 was degraded within 3 hours in nearly all the cases, except for S. aureus V8 protease due to a lack of acidic glutamates and aspartates in the sequence (FIG. 2 (top), lanes 4 and 9). Nevertheless, C10-KR8d was resistant to the five proteases, including trypsin, chymotrypsin, pancreas elastase, S. aureus V8 protease, and fungal proteinase K (FIG. 2 (bottom)). Thus, C10-KR8d is more stable than the L-form of C10-KR8. It appeared that the protease stability of C10-KR8d is superior to 17BIPHE2, which can be cleaved by trypsin, but not chymotrypsin, S. aureus V8 protease, and fungal proteinase K (Wang, et al. (2014) ACS Chem. Biol., 9:1997-2002).

### C10-KR8d Targets Bacterial Membranes

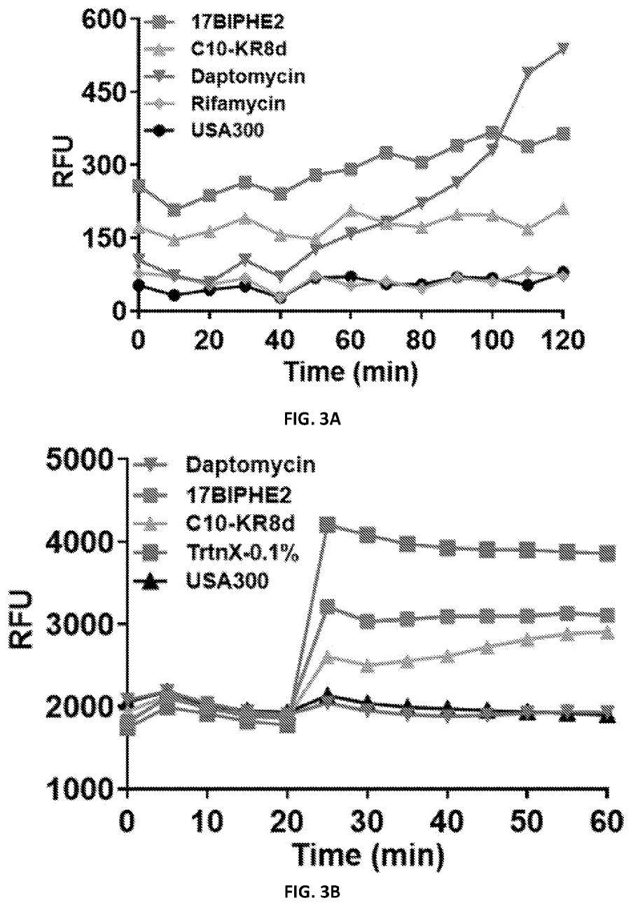

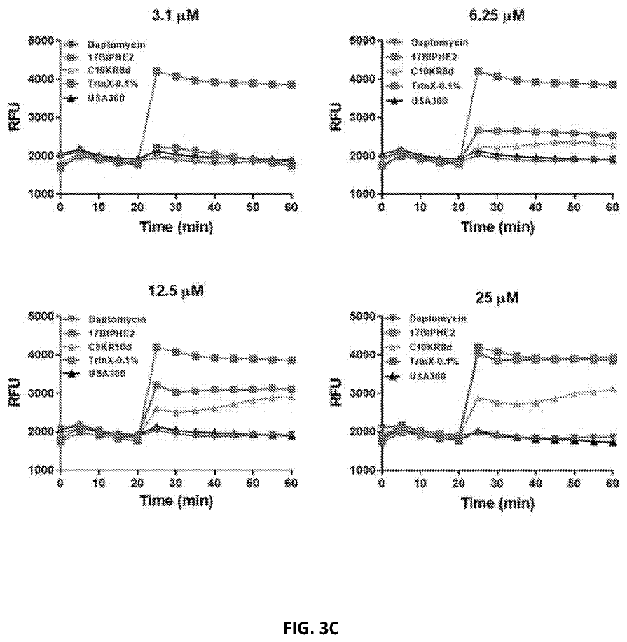

Similar MIC values for the L-and D-forms of C10-KR8d suggest membrane targeting (Table 1). To provide additional evidence for the membrane targeting of C10-KR8d, membrane permeation and depolarization experiments were conducted. Membrane permeation is indicated by the fluorescence increase of a nonmembrane permeable dye propidium iodide due to binding to bacterial DNA (Wang, et al. (2014) ACS Chem. Biol., 9:1997-2002). This can only occur when bacterial membranes are compromised by membrane active antibiotics such as daptomycin (FIG. 3A). Similar to 17BIPHE2 (Wang, et al. (2014) ACS Chem. Biol., 9:1997-2002), C10-KR8d was able to permeate the membranes of S. aureus USA300 LAC. As a negative control, rifamycin, which inhibits bacterial DNA-dependent RNA synthesis, was unable to achieve the same effect. The membrane potential is essential for the normal physiology of bacteria. Some chemicals, such as Triton™ X-100, can destroy this potential, leading to membrane depolarization (FIG. 3C). The same order was observed for the depolarization ability: Triton™ X-100>17BIPHE2>C10-KR8d>daptomycin at different concentrations, although the effect was small at 3.1 μM. The curves started to separate at 6.25 μM but were best resolved at 12.5 μM (FIG. 3B). At 25 μM, the depolarization effect of 17BIPHE2 approached that of Triton™-X100 (FIG. 3C). C10-KR8d could also achieve this although at a lower level than that of Triton™ X-100. As in the case of membrane permeation (FIG. 3A), C10-KR8d was also less efficient than 17BIPHE2 in membrane depolarization (FIG. 3B). All these experiments support that C10-KR8d targets the membranes of MRSA.

### Conformation of Small Lipopeptides

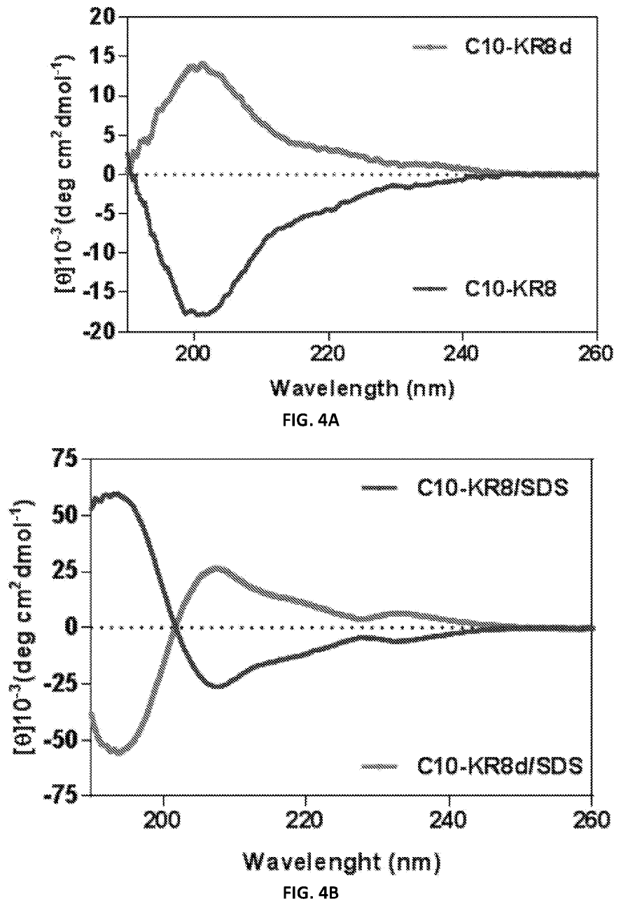

It was also determined what conformation C10-KR8 might adopt after membrane binding. FIG. 4 shows the CD spectra of both the free and bound peptides. In the 10 mM phosphate buffer (PBS), a negative band at 200 nm was observed for C10-KR8 (L-form). As anticipated, a mirror image with a positive band at ˜200 nm was generated by C10-KR8d (D-form). These spectra indicate that the peptide was randomly coiled under this condition. The mirror image of the two forms of the same peptide validated the high quality of synthetic peptides. Next, the CD spectra of these two forms of peptides were measured in the presence of membrane-mimetic micelles of sodium dodecyl sulfate (SDS). SDS is a useful model since similar helical structures were obtained for human cathelicdin LL-37 bound to SDS, dioctanoylphosphatidylglycerol (D8PG), or lipopolysaccharides (LPS) by multidimensional NMR spectroscopy (Wang, G. (2008) J. Biol. Chem., 283:32637-32643). For the L-form of C10-KR8, a positive band at ˜195 and a negative band at 208 nm are reminiscent of the well-known CD spectra of helical proteins (Wang, et al. (2005) J. Biol. Chem., 280:5803-5811; Sorensen, et al. (2003) J. Biol. Chem., 278:28540-6). The fact that the band at 222 nm was less evident implies a weak helix, probably due to a short peptide length with merely eight amino acids. Likewise, a mirror image for the D-form of C10-KR8 bound to SDS micelles was observed. These results indicate that the helical structure was retained for C10-KR8 in association with bacterial membranes.

Resistance Development of S. aureus to Small Lipopeptide

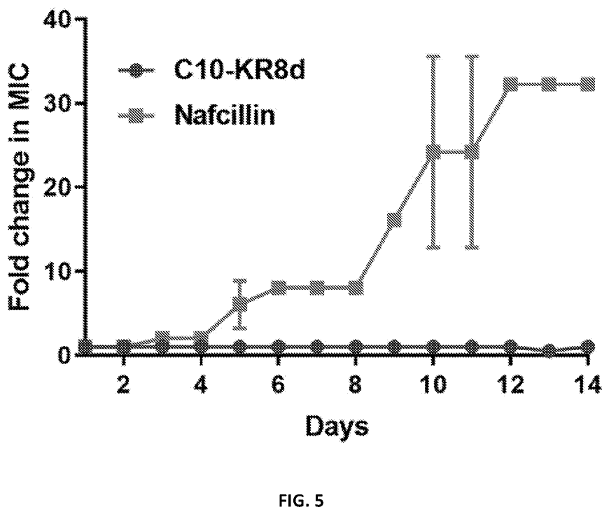

Membrane targeting would make it more difficult for bacteria to develop resistance (Mookherjee, et al. (2020) Nat. Rev. Drug Discovery 19:311-332). To illustrate this, a multiple passage experiment was conducted (Mishra, et al. (2019) Proc. Natl. Acad. Sci., 116:13517-13522). While the MIC of nafcillin increased by over 30 fold, no changes were observed for C10-KR8d in 14 days, indicating no resistance development of S. aureus USA300 LAC to this peptide (FIG. 5).

Antimicrobial Susceptibility of the S. aureus Mutants to (10-KR8d

The fact that MRSA did not develop resistance to C10-KR8d does not indicate a lack of bacterial response. Recently, two dozens of response genes have been identified from the Nebraska Transposon Mutant Library of S. aureus USA300 in the presence of LL-37 and its peptides (Golla, et al. (2020) ACS Infect. Dis., 6:1866-1881). As C10-KR8d contains part of the core antimicrobial sequence of LL-37, antimicrobial susceptibility of these transposon mutants were compared to the new peptide designed here. The bacterial strains as well as MIC values are provided in Table 6. Among the tested strains, over half of the S. aureus mutant strains were found to be susceptible (inhibited at a sub-MIC). These genes contribute to the response of MRSA to C10-KR8d (Golla, et al. (2020) ACS Infect. Dis., 6:1866-1881). They include the mutants for the major antimicrobial sensing system (MprF, GraS, GraR, and VraF) of S. aureus, potassium uptake protein (TrkA), and lipoprotein signal peptidase (LspA). The major antimicrobial sensing system can modify the membrane surface with a lysine via the membrane enzyme MprF, making MRSA less susceptible to cationic antimicrobial peptides (Falord, et al. (2012) Antimicrob. Agents Chemother., 56:1047-1058). The potassium uptake protein can modulate the membrane potential, again playing a role in bacterial susceptibility (Gries, et al. (2016) mSphere 1:e00125-16). As another example, LspA is found to be essential for resistance of Mycobacterium tuberculosis to malachite green (Banaei, et al. (2009) Antimicrob. Agents Chemother., 53:3799-3802). These genes may work together to reduce the likelihood of being killed by cationic AMPs. Since most of the LL-37 susceptible genes also respond to C10-KR8d, the results indicate that this newly discovered small lipopeptide acts like a miniature LL-37 peptide.

TABLE 6
Antimicrobial Susceptibility of the Transposon Mutants of S. aureus USA300 to C10-KR8d, a Miniature
lipoLL-37 Peptide. Mutants found in response to human cathelicidin LL-37 and its major antimicrobial
peptides GF-17 and 17BIPHE2 (Golla, et al. (2020) ACS Infect. Dis., 6: 1866-1881).

| | Gene | | MIC | |
|---|---|---|---|---|
| Gene ID | name | Potential function | (μM) | Susceptible? |
| SAUSA300 | JE2 | Wild type (WT) | 3.1 | Reference |
| SAUSA300_1962 | | phiPVL ORF39-like protein | 1.6-3.1 | Yes |
| SAUSA300_1255 | mprF | Oxacillin resistance-related FmtC protein | 1.6 | Yes |
| SAUSA300_0829 | lipA | Lipoyl synthase | 3.1 | No |
| SAUSA300_1036 | | RNA methyltransferase | 1.6-3.1 | Yes |
| SAUSA300_1465 | | 2-oxoisovalerate dehydrogenase, E1 component, beta subunit | 3.1 | No |
| SAUSA300_0186 | argC | N-acetyl-gamma-glutamyl-phosphate reductase | 3.1 | No |
| SAUSA300_0988 | trkA | Potassium uptake protein | 1.6 | Yes |
| SAUSA300_1089 | lspA | lipoprotein signal peptidase | 1.6 | Yes |
| SAUSA300_1515 | | ABC transporter permease | 1.6-3.1 | Yes |
| SAUSA300_1967 | | conserved hypothetical phage protein | 3.1 | No |
| SAUSA300_1228 | thrB | homoserine kinase | 1.6-3.1 | Yes |
| SAUSA300_0394 | | FAD/NAD(P)-binding Rossmann fold superfamily protein | 3.1 | No |
| SAUSA300_0646 | graS | Sensor histidine kinase | 1.6 | Yes |
| SAUSA300_0645 | graR | DNA-binding response regulator | 1.6 | Yes |
| SAUSA300_1865 | vraR | DNA-binding response regulator | 1.6 | Yes |
| SAUSA300_0647 | vraF | ABC transporter ATP-binding protein | 1.6-3.1 | Yes |
| SAUSA300_1336 | | Hypothetical protein | 3.1 | No |
| SAUSA300_1898 | | Conserved hypothetical protein | 3.1 | No |
| SAUSA300_1037 | pheS | Phenylalanyl-tRNA synthetase subunit alpha | 1.6 | Yes |
| SAUSA300_1503 | | Putative competence protein ComGB | 3.1 | No |
| SAUSA300_1867 | | Conserved hypothetical protein | 3.1 | No |
| SAUSA300_1097 | pyrF | orotidine 5′-phosphate decarboxylase | 3.1 | No |

No = no MIC changes of C10-KR8d against the WT and the mutant S. aureus strains measured in 50% TSB.

### In Vitro and In Vivo Toxicity of the LL-37 Derived Lipopeptides

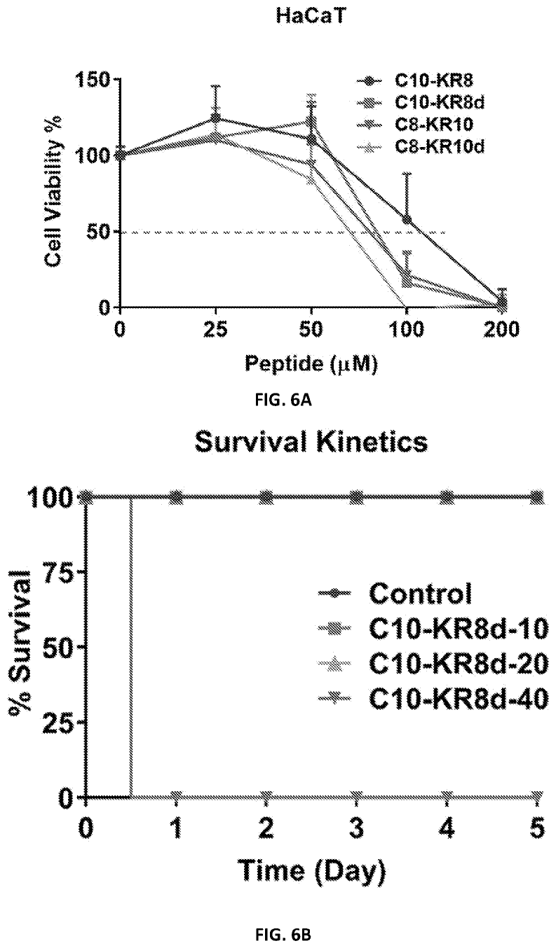

C10-KR8 showed poor hemolysis (HC₅₀300 μM in Table 1). To further evaluate peptide toxicity, human HaCaT cells were treated with the peptides at multiple doses and the lethal dose where 50% cells (TC₅₀) were dead was estimated (FIG. 6A). C10-KR8d was slightly more toxic than C10-KR8. The toxicity of the L-and D-forms of C8-KR10 was also compared. Again, C8-KR10 was slightly less toxic than C8-KR10d. These results indicate that the D-form was slightly more toxic than the L-form with TC₅₀ between ˜75 and ˜100 μM. Such toxic concentrations to skin and red blood cells are much higher than the MIC values of C10-KR8d at 3.1-6.2 μM (Table 4).

Peptide toxicity in mice was also tested. When C57BL/6 mice were injected with C10-KR8d intraperitoneally (i.p.) at 10 or 20 mg/kg per mouse, all mice survived during 5 days observation (FIG. 6B). However, all mice died in a day at a peptide dose of 40 mg/kg. This study indicates the maximal tolerated dose (MTD) is 20 mg/kg or more (but less than 40 mg/kg).

### Systemic in Vivo Efficacy of the Miniature LL-37 Peptide

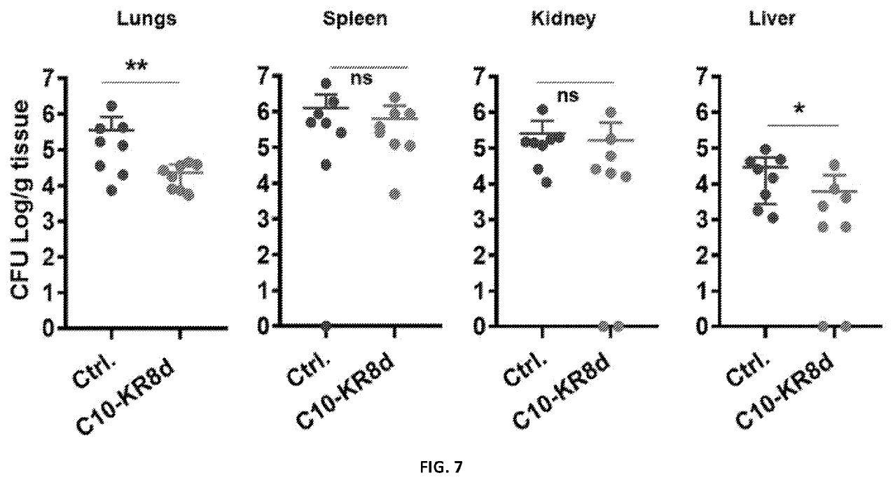

Since C10-KR8d has numerous drug-like properties (Table 4), its systemic efficacy in vivo was investigated. Neutropenic mice, generated by two injections of cyclophosphamide prior to infection, are widely used for this purpose since the effects of immune cells are minimized (Mishra, et al. (2019) Proc. Natl. Acad. Sci., 116:13517-13522; Radzishevsky, et al. (2007) Nat. Biotechnol., 25:657-9). On the basis of with other peptides and antibiotics controls, a bacterial inoculum of 2×10⁶ colony forming units (CFU) was selected to establish the S. aureus USA300 LAC infection (Mishra, et al. (2019) Proc. Natl. Acad. Sci., 116:13517-13522; Lakshmaiah et al. (2020) Proc. Natl. Acad. Sci., 117:19446-19454). The infected mice were treated with C10-KR8d at 5 mg/kg intraperitoneally. Compared to the untreated group, there was a significant CFU decrease in mouse lung and liver (˜0.5-1 log) but not in spleen and kidney (FIG. 7).

### Topical Antibiofilm Efficacy in a Catheter Murine Model

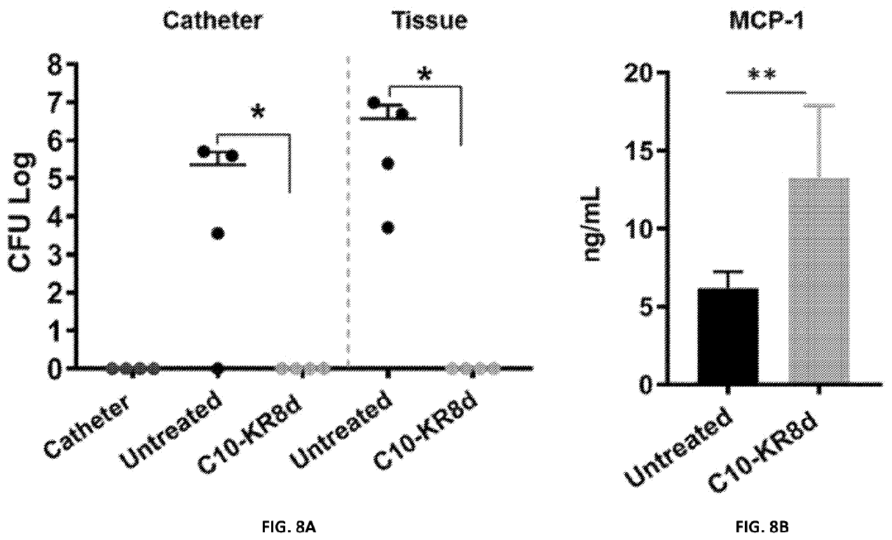

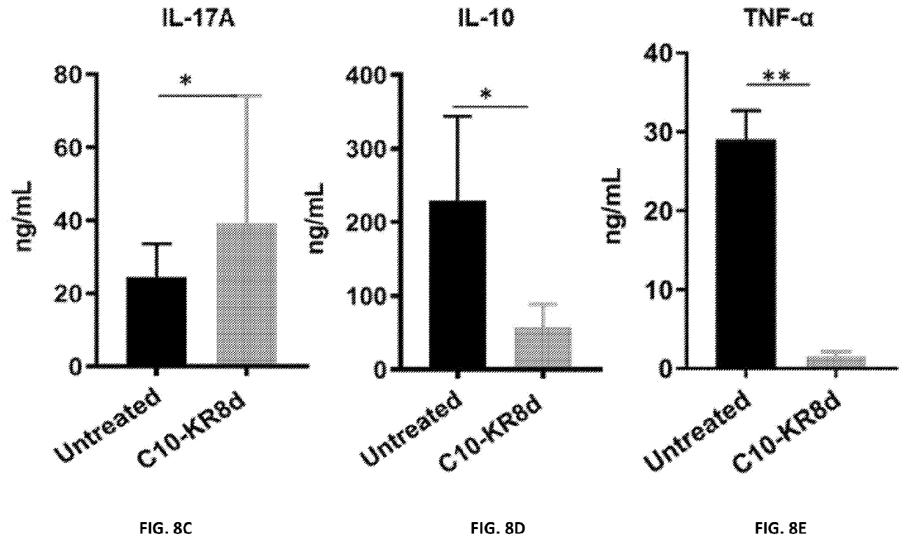

Preformed biofilms associated with medical devices are notoriously difficult to eliminate by conventional antibiotics. Therefore, a preferred strategy is to prevent biofilm formation. The antibiofilm capability of C10-KR8d was tested in an established catheter model (Wang, et al. (2014) ACS Chem. Biol., 9:1997-2002; Heim, et al. (2014) Methods Mol. Biol., 1106:183-191; Menousek, et al. (2012) Int. J. Antimicrob. Agents 39:402-406). In this experiment, catheters were inserted under the skin of the mouse flank; the mice were infected and treated by injecting the peptide into the lumen and around the catheter. After 3 days, mice were euthanized and the S. aureus CFUs on each catheter and its surrounding tissue were determined and presented in FIG. 8A. As anticipated, S. aureus USA300 LAC was not detected in the uninfected catheter. LL-23 V9, an LL-23 peptide analog derived from the N-terminus of LL-37, which is inactive against S. aureus, was unable to cause any CFU decrease in mice (Wang, et al. (2014) ACS Chem. Biol., 9:1997-2002). Therefore, a clear decrease in bacterial burden between the untreated and treated animal groups indicates an excellent efficacy of C10-KR8d (Saporito, et al. (2018) J. Pept. Sci., 8:e3080). The efficacy of one time single dose treatment of C10-KR8d was superior to 17BIPHE2, which achieved a similar effect after three single dose treatments in 3 days (Wang, et al. (2014) ACS Chem. Biol., 9:1997-2002).

One of the advantages of AMPs is their ability to curb invading pathogens by multiple mechanisms, making it difficult for bacteria to develop resistance (Nizet, et al. (2001) Nature 414:454-7; Putsep, et al. (2002) Lancet 360:1144-1149; Scott, et al. (2002) J. Immunol., 169:3883-91; Wang, G. (2014) Pharmaceuticals 7:545-594; Agerberth, et al. (1995) Proc. Natl. Acad. Sci., 92:195-9; Durr, et al. (2006) Biochim. Biophys. Acta, Biomembr. 1758:1408-1425; Vandamme, et al. (2012) Cell. Immunol., 280:22-35). For example, AMPs may also regulate cytokines that recruit immune cells. To get evidence, the levels of cytokines in the surrounding tissues of catheters were measured with and without C10-KR8d treatment. An up-regulation of MCP-1/CCL2 (monocyte chemoattractant protein-1) (FIG. 8B) and IL-17A (FIG. 8C) on day 3 after peptide treatment suggests the recruitment of monocytes (Wang, et al. (2014) ACS Chem. Biol., 9:1997-2002) and neutrophils (Cho, et al. (2010) J. Clin. Invest., 120:1762-73), respectively, which could partially contribute to the clearance of MRSA infection. Meanwhile, a decrease in the levels of IL-10 (FIG. 8D) and TNFα (FIG. 8E) could reduce inflammation to avoid potential tissue damage (Putsep, et al. (2002) Lancet 360:1144-1149; Leech, et al. (2017) J. Immunol., 198:2352-2365).

There are two major methods for drug discovery: rational design and library screening (Mishra, et al. (2017) Curr. Opin. Chem. Biol., 38:87-96). The structure-based approach is widely utilized when a molecular target has been identified. Both methods have been utilized for human cathelicidin LL-37 (Wang, et al. (2019) Adv. Exp. Med. Biol., 1117:215-240; Braff, et al. (2005) J. Immunol., 174:4271; Nagant, et al. (2012) Antimicrob. Agents Chemother., 56:5698-708; den Hertog, et al. (2006) Biol. Chem., 387:1495-502; Nan, et al. (2012) Peptides 35:239-47; Nell, et al. (2006) Peptides 27:649-60; Li, X., Li, et al. (2006) J. Am. Chem. Soc., 128:5776-85; Wang, et al. (2012) Antimicrob. Agents Chemother., 56:845-856; Wang, G. (2008) J. Biol. Chem., 283:32637-32643; Wang, et al. (2014) ACS Chem. Biol., 9:1997-2002; Narayana, et al. (2019) Biochim. Biophys. Acta, Biomembr., 1861:1592-1602). The present study differs from the traditional random library in that a systematic approach was used to generating numerous peptides followed by conjugation with various fatty acids. Since some lipopeptides (e.g., daptomycin and colistin) are already in clinical use as essential antibiotics, KR12 segments were conjugated with fatty acids. The 2D molecular array allowed for the identification of small LL-37 lipopeptides with high selectivity, stability, and antimicrobial robustness. Two less hemolytic peptides were found: C10-KR8 and C8-KR10 (Table 1). A recent study found an optimal peptide when KR12 was conjugated with C8 (Kamysz, et al. (2020) Int. J. Mol. Sci. 21:887). Notably, the goal was not to design minimal lipopeptides (as short as two amino acids) that no longer resemble LL-37. In fact, even shorter peptides such as KRIW do not offer advantages in terms of both antimicrobial activity and cell selectivity (Table 1). In agreement, ultrashort lipopeptides (e.g., <10 aa) may have limited cell selectivity (Makovitzki, et al. (2006) Proc. Natl. Acad. Sci., 103:15997-6002; Mishra, et al. (2015) RSC Adv., 5:59758-59769). Depending on the peptide sequence, there may be an optimal combination in terms of both peptide length and fatty acid chain length. The present systemic study identified a selective zone (10 carbon fatty acid×8mer peptides or 8 carbon fatty acid×10mer peptides) (Table 1), which may be utilized to design new lipopeptides.

C10-KR8d (made of D-amino acids), which is even more selective than C8-KR10 (Table 1), showed robust in vitro antibacterial activity under different media conditions (Table 4). In addition, the antibacterial activity of this peptide did not change in different media or with the content of TSB, whereas the antibacterial activity of both KR12 and human LL-37 was compromised in 100% TSB, especially against MRSA (Table 3). It appears that KR12 also contains the minimal LL-37 sequence required to interact with unidentified components in TSB. It is notable that C10-KR8 (made of L-amino acids) could lose activity in the presence of human serum (Table 4). The bead binding studies revealed that many mouse plasma proteins could bind to the L-form, but very few were associated with the D-form peptide. This is interesting and indicates that protein binding depends on the peptide chiral property. Human LL-37 is known to bind to human serum protein apolipoprotein A-I, leading to a loss of its antibacterial activity in vitro (Wang, et al. (1998) J. Biol. Chem., 273:33115-8). Indeed, the proteomic study reveals numerous murine apolipoproteins (apo), including apoA-I, apoA-IV, apoC-I, and apoC-III that preferentially bind to the L-form of C10-KR8. Since C10-KR8 associates with apoA-I similar to LL-37, the results further support that C10-KR8 retains the minimal functional core region of LL-37. Also, the tendency of the L-form of the peptide to interact with multiple serum proteins provides one possible mechanism for its loss of efficacy in vivo, since the D-form showed an antimicrobial efficacy in certain murine organs (FIGS. 7, 8). In this case, the extent of serum binding of the peptide in vitro appears to be a useful indicator for in vivo efficacy. Notably, the L-and D-forms of the database-designed peptides showed similar systemic efficacy in the same mouse model, although their antibacterial activities were compromised by human serum to different extents in vitro (Mishra, et al. (2019) Proc. Natl. Acad. Sci., 116:13517-13522). Likewise, horine has a slightly higher tendency to associate with human serum than verine, but they are equally potent in eliminating MRSA in mice (Lakshmaiah et al. (2020) Proc. Natl. Acad. Sci., 117:19446-19454). It seems that serum binding may not be the sole reason for peptide activity loss. A peptide, when injected intravenously, may take advantage of the serum vehicle to reach different organs to kill pathogens.

Due to the challenging nature to develop peptides with systemic efficacy, the antimicrobial peptide field is currently focusing on topical applications. Wound healing and catheter are two commonly used models for this purpose. A potent antibiofilm peptide SAAP-148 derived from the C-terminal region of LL-37 (residues 13-36) has been identified (de Breij, et al. (2018) Sci. Transl. Med., 10:eaan4044). Notably, the peptide was effective in mice but not in rats (Dijksteel, et al. (2019) Ann. Clin. Microbiol. Antimicrob., 18:38). The efficacy of 17BIPHE2 and its analog in preventing biofilm formation in a catheter-associated mouse model as well as wound healing in mice has been demonstrated (Wang, et al. (2014) ACS Chem. Biol., 9:1997-2002; Narayana, et al. (2019) Biochim. Biophys. Acta, Biomembr., 1861:1592-1602; Dijksteel, et al. (2019) Ann. Clin. Microbiol. Antimicrob., 18:38). While SAAP-148 consists of 24 amino acids, 17BIPHE2 is 17-residue long. Thus, the identification of a miniature LL-37 like peptide, C10-KR8d, here, with merely eight residues, further shortened the peptide length, reducing the cost of synthesis. It is remarkable that C10-KR8d (single dose treatment at 15 mg/kg) was able to achieve the same efficacy as 17BIPHE2 (three single dose treatments in 3 days at 3×15 mg/kg) in the catheter-associated biofilm model. The results underscore the fact that these small LL-37 lipopeptides obtained here are potent candidates to prevent biofilm infections of antibiotic-resistant pathogens such as MRSA.

Finally, it is useful to note that S. aureus did not develop resistance to C10-KR8d in a multiple passage experiment (FIG. 5). Using the Nebraska Transposon Mutant Library of S. aureus USA300 (Fey, et al. (2013) mBio 4:e00537-12), multiple genes were identified that respond to the action of C10-KR8d (Table 6). MRSA became more susceptible in the absence of such genes as indicated by reduced MIC values evaluated under the same conditions. These genes, as well as unidentified genes of S. aureus, may work together as a network to compromise the effect of antimicrobials eventually. A combination of the newly designed peptide with inhibitors that target these genes may slow down the development of Staphylococcal resistance.

LL-37 is a human cathelicidin in the innate immune system important for fighting infections via both direct killing and immune modulation. This study advanced LL-37 engineering and identified a miniature LL-37 lipopeptide shorter than KR12 (the smallest antimicrobial peptide) via a systematic 2D molecular array. The sequence of the newly identified peptide has been shortened to the extent that its antimicrobial activity was not influenced by different media or the TSB media contents. The proteomic studies indicate that C10-KR8 also retained some properties of LL-37. Indeed, the L-form of C10-KR8 lost activity in vitro due to association with numerous serum proteins. However, only a few proteins were bound to the peptide synthesized in D-amino acids (C10-KR8d). This D-peptide tolerated physiological salts, pH, and serum. Such antimicrobial robustness may explain the systemic efficacy observed for the D-form of the lipoLL-37 peptide in mice. Moreover, the miniature LL-37 like peptide, C10-KR8d, identified here, showed an excellent activity in preventing biofilm formation of MRSA in a catheter-associated mouse model, thereby offering new therapeutic opportunities. In addition, the identification of the susceptible genes of S. aureus in the presence of C10-KR8d can lead to combined treatment to better combat such resistant pathogens.

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

1. An antimicrobial lipopeptide comprising:
a peptide which is a truncation of the amino acid sequence KRIXQRIKDFLR (SEQ ID NO: 8), wherein X is W or V; and
a fatty acid,
wherein said fatty acid is conjugated to the N-terminus of the peptide.

2. The antimicrobial lipopeptide of claim 1, wherein said peptide comprises the 4, 5, 6, 7, 8, 9, 10, or 11 N-terminal amino acids of SEQ ID NO: 8.

3. The antimicrobial lipopeptide of claim 1, wherein said peptide comprises the amino acid sequence KRIW (SEQ ID NO: 6) or KRIV (SEQ ID NO: 9).

4. The antimicrobial lipopeptide of claim 1, wherein said peptide comprises the amino acid sequence KRIWOR (SEQ ID NO: 5) or KRIVQR (SEQ ID NO: 10).

5. The antimicrobial lipopeptide of claim 1, wherein said peptide comprises the amino acid sequence KRIWQRIK (SEQ ID NO: 4) or KRIVORIK (SEQ ID NO: 11).

6. The antimicrobial lipopeptide of claim 1, wherein said peptide comprises the amino acid sequence KRIWQRIKDF (SEQ ID NO: 3) or KRIVORIKDF (SEQ ID NO: 12).

7. The antimicrobial lipopeptide of claim 1, wherein said fatty acid is a saturated fatty acid.

8. The antimicrobial lipopeptide of claim 1, wherein said fatty acid is a

9. The antimicrobial lipopeptide of claim 1, wherein said fatty acid is a C7-C11 fatty acid.

10. The antimicrobial lipopeptide of claim 1, wherein said fatty acid is a C8-C10 fatty acid.

11. The antimicrobial lipopeptide of claim 1, wherein said peptide comprises at least one D-amino acid.

12. The antimicrobial lipopeptide of claim 11, wherein all of the amino acids are D-amino acids.

13. The antimicrobial lipopeptide of claim 1, wherein said peptide is amidation.

14. The antimicrobial lipopeptide of claim 1, wherein said peptide and fatty acid are conjugated by an amide bond.

15. The antimicrobial lipopeptide of claim 1, wherein said peptide is KRIWORIKDF (SEQ ID NO: 3) or KRIVORIKDF (SEQ ID NO: 12) and said fatty acid is octanoic acid.

16. The antimicrobial lipopeptide of claim 1, wherein said peptide is KRIWORIK (SEQ ID NO: 4) or KRIVQRIK (SEQ ID NO: 11) and said fatty acid is decanoic acid.

17. A composition comprising at least one antimicrobial lipopeptide of claim 1 and at least one pharmaceutically acceptable carrier.

18. The composition of claim 16, further comprising at least one antibiotic.

19. A method for inhibiting a microbial infection in a subject in need thereof, said method comprising administering to said subject at least one antimicrobial lipopeptide of claim 1.

20. The method of claim 19, further comprising the administration of at least one additional antibiotic.

21. The method of claim 19, wherein said microbial infection is a staphylococcal infection.

22. The method of claim 19, wherein said microbial infection is a methicillin-resistant Staphylococcus aureus (MRSA) infection.

23. The method of claim 19, wherein said antimicrobial lipopeptide is administered topically.

24. A medical device or implant comprising at least one antimicrobial lipopeptide of claim 1 attached to the surface of the medical device or implant.


SEQUENCE LISTING (12 sequences)

SEQ ID NO: 1 - protein, 37 aa, Homo sapiens
LLGDFFRKSKEKIGKEFKRIVQRIKDFLRNLVPRTES

SEQ ID NO: 2 - protein, 12 aa, Artificial Sequence
  KR12
KRIWQRIKDFLR

SEQ ID NO: 3 - protein, 10 aa, Artificial Sequence
  KR12 peptide
KRIWQRIKDF

SEQ ID NO: 4 - protein, 8 aa, Artificial Sequence
  KR12 peptide
KRIWQRIK

SEQ ID NO: 5 - protein, 6 aa, Artificial Sequence
  KR12 peptide
KRIWQR

SEQ ID NO: 6 - protein, 4 aa, Artificial Sequence
  KR12 peptide
KRIW

SEQ ID NO: 7 - protein, 12 aa, Artificial Sequence
  KR12
KRIVQRIKDFLR

SEQ ID NO: 8 - protein, 12 aa, Artificial Sequence
  KR12 peptide
  VARIANT 4: Xaa = Trp or Val
KRIXQRIKDFLR

SEQ ID NO: 9 - protein, 4 aa, Artificial Sequence
  KR12 peptide
KRIV

SEQ ID NO: 10 - protein, 6 aa, Artificial Sequence
  KR12 peptide
KRIVQR

SEQ ID NO: 11 - protein, 8 aa, Artificial Sequence
  KR12 peptide
KRIVQRIK

SEQ ID NO: 12 - protein, 10 aa, Artificial Sequence
  KR12 peptide
KRIVQRIKDF